SEQUENCE LISTING (4 sequences)

SEQ ID NO: 1 - DNA, 20 bp, Artificial Sequence
  primer
ccgctcaggttctgctttta

SEQ ID NO: 2 - DNA, 19 bp, Artificial Sequence
  primer
ggctgaggaagctgaggag

SEQ ID NO: 3 - DNA, 34 bp, Artificial Sequence
  primer
gtaggacttattgtggtgtatacaattgcatttg

SEQ ID NO: 4 - DNA, 24 bp, Artificial Sequence
  primer
cagtatttccagagcagtgagcgc

(19) United States
(12) Patent Application Publication
(10) Pub. No.: US 2022/0064644 A1
(43) Pub. Date: Mar. 3, 2022

(54) METHODS FOR IDENTIFICATION OF GENETIC MODIFIERS AND FOR TREATING NUCLEOTIDE REPEAT DISORDER

(71) Applicants: TAIPEI MEDICAL UNIVERSITY, Taipei City (TW); NATIONAL YANG MING CHIAO TUNG UNIVERSITY, Hsinchu (TW); ACADEMIA SINICA, Taipei (TW); NATIONAL TAIWAN NORMAL UNIVERSITY

(72) Inventors: Bing-Wen SOONG, Taipei City (TW); Ueng-Cheng YANG, Taipei City (TW); Yu-Shuen TSAI, Taipei City (TW); Tzu-Hao CHENG, Taipei City (TW); Yi-Juang CHERN, Taipei (TW); Ming-Tsan SU, Taipei (TW); Yi-Ching CHANG, Taipei City (TW); Yan Hua LEE, Taipei (TW)

(21) Appl. No.: 17/461,876

(22) Filed: Aug. 30, 2021

Related U.S. Application Data
(60) Provisional application No. 63/071,903, filed on Aug. 28, 2020.

(51) Int. Cl. C12N 15/113; A61K 31/7115

(52) U.S. Cl. CPC C12N 15/113; A61K 31/7115

(57) ABSTRACT

The present disclosure relates to a method of identifying a genetic modifier of a nucleotide repeat disorder, comprising selecting from the subjects the late-onset subjects with higher nucleotide repeat load or the early-onset subjects with lower nucleotide repeat load and identifying one or more genetic modifiers delaying or promoting onset of a nucleotide repeat disorder. The present disclosure also relates to a method for treating or preventing a polyglutamine (polyQ) expansion disease in a subject in need of such treatment or prevention, comprising administering an effective amount of a PIAS1 variant or a recombinant nucleic acid molecule encoding the PIAS1 variant to the subject. The present disclosure also relates to a method for treating or preventing early symptoms onset of the polyglutamine expansion disease, a PIAS1 variant, comprising one or more sequence changes located in the C-terminal region of PIAS1 and a recombinant nucleic acid molecule encoding the PIAS1 variant as disclosed herein.

### CROSS REFERENCE TO RELATED APPLICATION

This patent application claims priority to and benefit of U.S. Provisional Application No. 63/071,903 filed Aug. 28, 2020. The application is incorporated by reference herein.

### FIELD OF THE DISCLOSURE

The present disclosure relates to a method for treating nucleotide repeat disorders, particularly to methods for identifying genetic modifiers and for treating nucleotide repeat disorders by genetic modifiers.

### SEQUENCE LISTING

The present disclosure contains sequences. An electronic Sequence Listing is submitted concurrently with this application under the name, “G4590-10000US_SeqListing_20210830” and is 2 KB in size.

In connection with the electronic Sequence Listing submitted concurrently herewith, the Applicant hereby states that the content of the electronically filed submission is in accordance with 37 C.F.R. § 1.821(e). The submission of the electronic Sequence Listing, in accordance with 37 C.F.R. § 1.821(g), does not include any new matter from what is listed in this Specification.

### BACKGROUND OF THE DISCLOSURE

Instability of repetitive DNA sequences within the genome is associated with a number of human diseases. The expansion of nucleotide repeats is recognized as a major cause for neurological and neuromuscular diseases. As the nucleotide repeat number grows in a specific gene, the growing triplet tract alters gene expression and/or function of the gene product. Expansion of the nucleotide repeats residing in a coding sequence of a gene typically produce a faulty protein, while expansion of a nucleotide repeat in a noncoding gene region has an impact on the gene expression, alters its splicing, or may influence aspects of antisense regulation.

For example, among the age-dependent protein aggregation disorders, many neurodegenerative diseases are caused by expansions of CAG repeats encoding polyglutamine (polyQ) tracts, including Huntington's disease (HD), spinal-bulbar muscular atrophy (SBMA), dentatorubral-pallidoluysian atrophy (DRPLA) and the spinocerebellar ataxias (SCA) types 1, 2, 3, 6, 7 and 17. Each of these disorders results from the expansion of a CAG repeat, coding for a glutamine tract that is present in the wild-type protein.

Some strategies have been provided for treating polyQ expansion diseases. US 20050277133A1 discloses a chemically synthesized double stranded short interfering nucleic acid molecule targeting a huntingtin (HTT) RNA for treating Huntington's disease. US20070270462A1 provides a method of treating a polyQ expansion disease using an 8-hydroxyquinoline compound. However, the conventional biologic therapy or the chemical agent both fail to achieve sufficient effects.

There is a need in the art for new detection and treatments of nucleotide repeat expansion diseases.

### SUMMARY OF THE DISCLOSURE

The present disclosure provides a method of identifying a genetic modifier of a nucleotide repeat disorder, comprising: (a) providing a length of one or more nucleotide repeats in samples obtained from subjects to obtain a nucleotide repeat load in genomes of the subjects; (b) clustering the subjects based on overall nucleotide repeat loads in the subject; (c) selecting from the subjects the late-onset subjects with higher nucleotide repeat load or the early-onset subjects with lower nucleotide repeat load; and (d) identifying one or more genetic modifiers delaying or promoting onset of a nucleotide repeat disorder.

In one embodiment, the method further comprises a step of administrating one or more genetic modifiers to a subject to treat or prevent the nucleotide repeat disorder.

In some embodiments of the disclosure, the nucleotide repeat is a trinucleotide repeat (TNR). Examples of the TNR include but are not limited to a CGG-repeat, a CTG-repeat, a GAA-repeat or a CAG-repeat.

In some embodiments of the disclosure, the nucleotide repeat disorder is a polyglutamine (polyQ) expansion disease. Examples of the nucleotide repeat disease include but are not limited to Huntington's disease (HD), spinocerebellar ataxias, spinal and bulbar muscular dystrophy (SBMA), or dentatorubral-pallidoluysian atrophy (DRPLA).

In some embodiments of the disclosure, the clustering is performed using Euclidean distance and Ward's method.

In some embodiments of the disclosure, the nucleotide repeat load is a CAG load, a CGG load, a CTG load or a GAA load.

The present disclosure also provides a computer system for identifying a genetic modifier of a nucleotide repeat disorder, comprising:
a database that is configured to store data of the length of one or more nucleotide repeats in samples obtained from subjects to obtain nucleotide repeat load in genomes of the subjects; and
one or more computer processors operatively coupled to the database, wherein the one or more computer processors are individually or collectively programmed to cluster the subjects based on overall nucleotide repeat loads in the subject; select from the subjects the late-onset subjects with higher nucleotide repeat load or the early-onset subjects with lower nucleotide repeat load; and identify one or more genetic modifiers delaying or promoting onset of a nucleotide repeat disorder.

The present disclosure also provides a non-transitory computer-readable medium comprising machine-executable instructions which, upon execution by one or more computer processors, perform the method of identifying a genetic modifier of a nucleotide repeat disorders as described herein.

The present disclosure provides a method for treating or preventing a polyglutamine (polyQ) expansion disease in a subject in need of such treatment or prevention, comprising administering an effective amount of a PIAS1 variant or mutant or a recombinant nucleic acid molecule encoding the PIAS1 variant or mutant to the subject.

In some embodiments of the disclosure, the PIAS1 variant or mutant comprises one or more amino acid changes located in the C-terminal region of wild type PIAS1. Examples of the PIAS1 variant or mutant include, but are not limited to S510G, A445T and T635M and one or more combinations thereof. In some embodiments of the disclosure, the PIAS1 variant or mutant is provided through the recombinant nucleic acid molecule encoding the PIAS1 variant or mutant.

In some embodiments of the disclosure, the method is for reducing accumulation of mutant polyQ proteins in the subject. Particularly, in some embodiments of the disclosure, the method is for lowering SUMOylation of mutant polyQ proteins in the subject. In some embodiments of the disclosure, the method is for de-stabilizing mutant polyQ proteins. In some embodiments of the disclosure, the method is for preventing mutant polyQ proteins aggregation and toxicity, for decrease of SUMO3-conjugation on mutant polyQ proteins, for reduction of foci formation and cell death, or for improving motor function.

Examples of the polyQ proteins include but are not limited to huntingtin (HTT) ataxin-1 (ATXN1), ataxin-2 (ATXN2), ataxin-3 (ATXN3), calcium voltage-gated channel subunit alphal A (CACNA1A), ataxin-7 (ATXN7), TATA box-binding protein (TBP), atrophin-1 (ATN1), or androgen receptor (AR).

In some embodiments of the disclosure, the method is for treating or preventing early symptoms onset of the polyglutamine expansion disease.

The present disclosure also provides a method for treating or preventing early symptoms onset of the polyglutamine expansion disease in a subject in need of such treatment or prevention, comprising administering an effective amount of an agent for diminishing the effect of wild type PIAS1 in the subject.

In some embodiments of the disclosure, the agent is a PIAS1 variant or mutant or a recombinant nucleic acid molecule encoding the PIAS1 variant or mutant as disclosed herein to the subject.

In some embodiments of the disclosure, the agent is an RNA interference agent (RNAi). Examples of the RNAi include but are not limited to a small inhibitory RNA (siRNA), a microRNA (miRNA), and a small hairpin RNA (shRNA).

The present disclosure also provides the PIAS1 variant or mutant, comprising one or more sequence changes located in the C-terminal region of PIAS1. In one embodiment, the PIAS1 variant or mutant comprises S510G, A445T or T635M or one or more combinations thereof.

The present disclosure provides the recombinant nucleic acid molecule encoding the PIAS1 variant or mutant as disclosed herein.

### BRIEF DESCRIPTION OF THE DRAWINGS

FIG. 1 shows the resultant dendrogram composed of three main clusters for a cohort of 361 SCA3 patients. The numbers of repeat expansions in both alleles of seven polyQ disease causing genes were used for measure of Euclidean distance between patients. A hierarchical clustering with Ward's method was subsequently performed. As a result, three main clusters were identified.

FIG. 2 shows the scatter plots of SCA3 patients of three different clusters. The x-axis represents the numbers of CAG repeat in patients' disease alleles and y-axis corresponds to the natural logarithm of patients' age at onset time. Each figure represents the overall patients of the same cluster.

FIG. 3 shows a decision tree of good discrimination for these three clusters. These three clusters can be well discriminated by simple rules. In a nutshell, patients of cluster 1 have a higher mean CAG load than the other two clusters, which may also imply a higher cellular burden of these patients.

FIGS. 4 (A) to (C) show that PIAS1 gene variant 3 confers a decrease of mutant ATXN3-mediated cell dead and protein aggregation. (FIG. 4 (A)) PIAS1 gene variants together with EGFP-ATXN3-28Q or EGFP-ATXN3-84Q were co-transfected into HeLa cells. 24-hour post transfection, cell lysates were collected as soluble and insoluble fractions. ATXN3 protein levels were analysis by Western blot, and β-actin served as internal control. The ATXN3 protein level in cells transfected with vector plasmid was set as 100%. (FIG. 4 (B)) EGFP-ATXN3-84Q was co-transfected with vector (vec), wild-type PIAS1 (WT) or PIAS1 gene variant 3 (v3) into ST14A cells. 48-hour post transfection, cell imaginings were acquired by fluorescence microscope. Quantification of cells with aggregation foci in GFP-positive cells. Data are presented as mean±SD. * p<0.05 by Student's t test. N=3. (FIG. 4 (C)) The effect of EGFP-ATXN3-84Q mediated cell death on ST14A cells expressing vector, wild-type PIAS1, or PIAS1 gene variant 3 was analyzed by trypan blue assay. Data are presented as mean±SD. * p<0.05, ** p<0.01, *** p<0.001 by Student's t test. N=3.

FIGS. 5 (A) to (C) show that PIAS1 promotes SUMO3 conjugation and insoluble form of mutant ATXN3. (FIG. 5 (A)) HeLa cells expressing HA-SUMO3 and ATXN3-84Q were transfected with different doses (1×, 2 μg; 2×, 4 μg) of PIAS1 shRNA construct. 24-hour post transfection, cells were treated with MG132 (40 μM) for 4 hours, followed by separation of cell lysates into soluble and insoluble fractions and then analyzed by Western blot. After f-actin normalization, the level of ATXN3-84Q in samples without PIAS1 shRNA knockdown was set as 100%. DMSO was included as the solvent control of MG132. Data were analyzed by one-way ANOVA with Dunnett's test. *P<0.05, **P<0.01, ***P<0.001. (FIG. 5 (B)) HeLa cells expressing ATXN3-84Q and PIAS1 shRNA were subjected to Cycloheximide chase assay for analysis ATXN3-84Q protein stability. ATXN3 protein levels were analysis by Western blot, and Q-actin served as internal control. The ATXN3 protein level in cells treated with CHX at 0 hour was set as 100%. (FIG. 5 (C)) Soluble fractions with MG132 treatment as described in (FIG. 5 (A)) were subjected to in vivo SUMOylation assay using ATXN3 antibody. Immuno-precipitates were then analyzed by Western blot using antibody against ATXN3 and HA-tagged SUMO3. The signal of SUMO3 conjugation on ATXN3-84Q in samples without PIAS1 shRNA knockdown was set as 100%. Quantified results were shown at right panel. Data were analyzed by one-way ANOVA with Dunnett's test. *P<0.05, **P<0.01, ***P<0.001.

FIGS. 6 (A) to (B) show that PIAS1 gene variant 3 causes a decrease of SUMOylation on mutant ATXN3. (FIG. 6 (A)) HeLa cells expressing HA-SUMO3 and EGFP-ATXN3-28Q or EGFP-ATXN3-84Q were transfected with wild-type PIAS1 (WT) or PIAS1 gene variant 3 (v3). 24-hour post transfection, supernatant of cell lysates was collected and subjected to in vivo SUMOylation assay using ATXN3 antibody. SUMO3 conjugated ATXN3 proteins were probed with HA antibody by Western blot. The SUMO3 conjugation signal of cells transfected with vector plasmid (vec) was set as 100%. Data are presented as mean±SD. * p<0.05 using Student's t test. N=3. (FIG. 6 (B)) ATXN3-28Q and 84Q were produced by in vitro Transcription/Translation and captured by ATXN3 antibody and protein G agarose beads. The ATXN3 protein were then subjected to in vitro SUMOylation assay with wild-type PIAS1 (WT) or PIAS1 gene variant 3 (v3). SUMOylated ATXN3 were analyzed by Western blot using SUMO3 and ATXN3 antibody.

FIGS. 7 (A) to (B) show that PIAS1 gene variant 3 interacts ordinarily with substrate ATXN3 proteins but defects in interacting with E2-ligase UBC9 in the presence of mutant ATXN3. (FIG. 7 (A)) Recombinant protein PIAS1 incubated with ATXN3 protein produced by In vitro transcription/translation, and ATXN3 were probed by ATXN3 antibody. Interaction between PIAS1 and ATXN3 were examined by Western blot. (FIG. 7 (B)) Interaction between PIAS1 and UBC9 were examined by GST pull-down assay, GST-Ubc9 were analyzed by Coomassie Blue staining and PIAS1 were analyzed by Western blot. GST protein served as negative control for UBC9, TNT ctrl served as negative control for ATXN3. The wild-type PIAS1 level was set as 100% when GST-Ubc9 served as internal control, and the relative levels present below. Data are presented as mean±SD. * p<0.05, **P<0.01 using Student's t test. N=3.

FIGS. 8 (A) to (E) show that reduction of post-translational modification of ATXN3 by PIAS1 achieves destablization of ATXN3-causing protein. Expression of ATXN3-84Q significantly reduced the expression of mCD8-GFP, while down-regulation of dPIAS increased the expression of mCD8-GFP (FIGS. 8 (A) and (B)). Knocking down dPIAS reduced the levels of both soluble and insoluble ATXN3-84Q proteins (FIG. 8s (C) and (D)). The motor function of SCA3 fly model expressing ATXN3-84Q was improved by silencing the expression of dPIAS (FIG. 8 (E)).

FIGS. 9 (A) and (B) show identification of rare gene variant(s) in LTA patients in the HD or SCA3 cohort. Natural logarithm of patient age of onset (log AO) and CAG repeat number in the expanded allele in patients with HD (FIG. 9 (A)) or SCA3 (FIG. 9 (B)). The line indicates the regression line calculated with log transformed data. Each dot represents a single patient. Earlier-than-average (ETA) and later-than-average (LTA) age of onset is the primary target of our genetic analysis. AAO: average age-at-onset; AO: age-at-onset.

FIGS. 10 (A) and (B) show that expression of PIAS1^{S510G} leads to a diminished accumulation of insoluble mHTT and a lower level of SUMO-modification of mHTT than wild-type PIAS1. (FIG. 10 (A)) HEK293 T cells were transfected with PIAS1^{WT} or PIAS1^{S510G} and Q₂₅-HTT_{EX1} or Q₁₀₉-HTT_{EX1} at a 3:1 ratio for 48 hrs and harvested for a filter trap assay. (FIG. 10 (B)) In vitro SUMOylation assay using purified E1 SUMO activating enzyme, E2 SUMO conjugating enzyme, SUMO-2-GG protein, purified GST-Q₄₃HTT_{EX1} and 6×His-PIAS1 (wild-type PIAS1 or PIAS1^{S510G}) The SUMOylation reaction was performed at 37° C. for 1 hr, and harvested for Western blot analyses. The arrowheads mark unmodified and SUMO-modified GST-Q₄₃HTT_{EX1}, respectively. The data are presented as the mean±SEM (N=3). *p<0.05, **p<0.01, ***p<0.001 by unpaired t test.

FIGS. 11 (A) and (B) show that PIAS1^{S510G} interacts with HTT proteins less effectively than wild-type PIAS1. HEK293T cells were transfected with the indicated construct for 48 hrs and harvested for pull-down assays. The indicated lysates (1 mg) were incubated with purified recombinant GST, GST-Q₂₅-HTT_{EX1} protein (FIG. 11 (A)) or GST-Q₄₃-HTT_{EX1} protein (FIG. 11 (B)) as indicated for 60 min at 4° C. to allow complex formation. GST served as a negative control. The protein complexes were analyzed by Western blotting. The amount of PIAS1 variant was normalized to the corresponding bait (GST-Q₂₅-HTT_{EX1} protein or GST-Q₄₃-HTT_{EX1}). The data are presented as the mean±SEM (N=3). **p<0.01 and ***p<0.001 by unpaired t test.

FIGS. 12 (A) to (C) show that S/T-rich region of PIAS1 binds to HTT. (FIG. 12 (A)) Schematic diagram of the HA-tagged PIAS1 mutant constructs for the pull-down assay shown in panel B. (FIG. 12 (B)) HEK293T cells were transfected with the indicated construct for 48 hrs and harvested for pull-down assays. The indicated lysates (1 mg) were incubated with purified recombinant GST or GST-Q₂₅-HTT_{EX1} protein as indicated for 60 min at 4° C. to allow complex formation. GST served as a negative control. The protein complexes were analyzed by Western blotting. The amount of PIAS1-domain-deletion mutant was normalized to the corresponding bait (GST-Q₂₅-HTT_{EX1} protein). The data are presented as the mean±SEM (N=4). **p<0.001 and ****p<0.0001 by one-way ANOVA. (FIG. 12 (C)) Purified recombinant 6×His-S/T rich region only of PIAS1 was incubated with purified recombinant GST or GST-Q₂₅-HTT_{EX1} protein for 60 min at 4° C. GST served as a negative control. The protein complexes were analyzed by Western blotting. The red arrowhead and yellow arrowhead mark the GST-Q₂₅-HTT_{EX1} and GST proteins, respectively. The data shown here represent three independent experiments.

FIG. 13 shows that phosphorylation of the Ser⁵¹⁰ residue of PIAS1 modulates its binding affinity to HTT. HEK293T cells were transfected with the indicated construct for 48 hrs and harvested for pull-down assays. The indicated lysates (1 mg) were incubated with purified recombinant GST or GST-Q₂₅-HTT_{EX1} protein as indicated for 60 min at 4° C. to allow complex formation. GST served as a negative control. The protein complexes were analyzed by Western blot analysis. The amount of PIAS1 mutant was normalized to the corresponding bait (GST-Q₂₅-HTT_{EX1} protein). The data are presented as the mean±SEM (N=3). *p<0.05 and **p<0.01 by unpaired t test.

FIGS. 14 (A) to (E) show that expression of Pias1^{S510G} moderates HD symptoms in R6/2 mice. Results from body weight (FIG. 14 (A)), grip strength (FIG. 14 (B)), limp clasping (FIG. 14 (C)), and pole test (FIG. 14 (D)), and survival (FIG. 14 (E)) measures were assessed. The data are presented as the means±SEM (n=9-13 animals per group). *, #p<0.05, **, ##p<0.01, and ****, ####p<0.0001 by two-way ANOVA. *Specific comparison between WT and R6/2 mice in each condition; #Specific comparison between mice expressing Piasl gene variants (WT/WT vs. S510G/S510G) under each condition. (FIG. 14(E)) Specific comparison between HD/Pias1^{WT/WT} vs. HD/Pias1^{S510G/S510G} (P=0.011; log-rank test).

FIGS. 15 (A) to (D) show that expression of Pias1^{S510G} leads to reduced accumulation of mHTT in R6/2 mice. (FIG. 15 (A)) The amount of insoluble mHTT in the striatal lysates was analyzed using a filter trap assay. Insoluble aggregates retained on the nitrocellulose membrane were detected with an anti-HTT antibody (Habe-1) selectively recognizing the oligomeric form of mHTT. The data are presented as the mean±SEM (N=3). *p<0.05 by unpaired t test. (FIGS. 15 (B) to (D)) Brain sections were subjected to immunofluorescence staining to determine the amount of mHTT aggregates (HTT) or SUMO-2/3 (green) in the nuclei (Hoechst). The data are presented as the means±SEM (n=6 animals per group). *p<0.05 by unpaired t test. Scale bar: 20 pm.

FIGS. 16 (A) and (B) show that expression of Pias1^{S510G} leads to reduced SUMO-modified mHTT in R6/2 mice. Brain sections were subjected to proximity ligation assay (PLA) to determine the amount of SUMO-modified mHTT using anti-HTT and anti-SUMO-2/3 antibodies. The data are presented as the means±SEM (n=6 animals per group). *p<0.05 by unpaired t test. Scale bar: 20 pm.

### DETAILED DESCRIPTION OF THE DISCLOSURE

### Definitions

Unless defined otherwise, technical and scientific terms used herein have the same meaning as commonly understood by one of ordinary skill in the art to which this disclosure pertains.

The articles “a” and “an” are used herein to refer to one or to more than one (i.e., to at least one) of the grammatical object of the article. By way of example, “an element” means one element or more than one element.

As used herein, the term “and/or” is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “polypeptide” is intended to encompass a singular “polypeptide” as well as plural “polypeptides,” and refers to a molecule composed of monomers (amino acids) linearly linked by peptide bonds (also known as amide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and unless specifically stated otherwise the term “polypeptide” can be used instead of, or interchangeably with any of these terms. The term “polypeptide” is also intended to refer to the products of post-expression modifications of the polypeptide, including, without limitation, glycosylation, acetylation, phosphorylation, amidation, derivatization by known protecting/blocking groups, proteolytic cleavage, or modification by non-standard amino acids. A polypeptide can be derived from a natural biological source or produced by recombinant technology, but is not necessarily translated from a designated nucleic acid sequence. Thus, it can be generated in any manner, including by chemical synthesis.

As used herein, the term “protein” refers to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

As used herein, the term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Further, the term nucleotide also includes those species that have a detectable label, such as for example a radioactive or fluorescent moiety, or mass label attached to the nucleotide.

As used herein, the term “polynucleotide” refers to polymers of nucleotides, and includes but is not limited to DNA, RNA, DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The term “polynucleotide” is also intended to encompass a singular nucleic acid as well as plural nucleic acids, and refers to an isolated nucleic acid molecule or construct, e.g., messenger RNA (mRNA) or plasmid DNA (pDNA). A polynucleotide can comprise a conventional phosphodiester bond or a non-conventional bond (e.g., an amide bond, such as found in peptide nucleic acids (PNA)). A polynucleotide can be single stranded or double stranded.

As used herein, the term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a polypeptide subunit contained in a vector is considered isolated as disclosed herein. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “expression” refers to a process by which a gene produces a biochemical, for example, a polynucleotide or a polypeptide. The process includes any manifestation of the functional presence of the gene within the cell including, without limitation, gene knockdown as well as both transient expression and stable expression. It includes, without limitation, transcription of the gene into messenger RNA (mRNA), and the translation of such mRNA into polypeptide(s). It also includes, without limitation, transcription of the gene into an RNA molecule that is not translated into a polypeptide but is capable of being processed by cellular RNAi mechanisms. If the final desired product is a biochemical, expression includes the creation of that biochemical and any precursors. Expression of a gene produces a “gene product.” As used herein, a gene product can be either a nucleic acid, e.g., an RNA produced by transcription of a gene or a polypeptide that is translated from an mRNA transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the term “siRNA” refers to small (or short) interfering RNA (or alternatively, silencing RNA) duplexes that are capable of inducing the RNA interference (RNAi) pathway. These molecules can vary in length (generally between 18-30 base pairs) and contain varying degrees of complementarity to their target mRNA in the antisense strand. Some, but not all, siRNA have unpaired overhanging bases on the 5′ or 3′ end of the sense strand and/or the antisense strand. The term “siRNA” includes duplexes of two separate strands, as well as single strands that can form hairpin structures comprising a duplex region.

As used herein, the phrase “gene silencing” refers to a process by which the expression of a specific gene product is lessened or attenuated. Silencing of a gene does not require that the expression or presence of the gene product is completely absent.

As used herein, the terms “patient,” “subject,” “individual,” and the like are used interchangeably, and refer to any animal, including any vertebrate or mammal, and, in particular, a human, and can also refer to, e.g., as an individual or patient.

As used herein, the term “trinucleotide repeat disorder” refers to a set of genetic disorders caused by trinucleotide repeat expansion, a kind of mutation in which repeats of three nucleotides (trinucleotide repeats) increase in copy numbers until they cross a threshold above which they become unstable. In one embodiment, the repeated trinucleotide, or codon, is CAG. In a coding region, CAG codes for glutamine (Q), so CAG repeats result in a polyglutamine tract. These diseases are commonly referred to as polyglutamine (or polyQ) expansion diseases.

As used herein, the term “modifier gene” refers to a gene that influences the disease expression and severity, influence a number of genetic diseases.

As used herein, “treating” or “treatment” of a state, disorder or condition includes: (1) preventing or delaying the appearance of clinical or sub-clinical symptoms of the state, disorder or condition developing in a mammal that may be afflicted with or predisposed to the state, disorder or condition but has not yet experienced or displayed clinical or subclinical symptoms of the state, disorder or condition; and/or (2) inhibiting the state, disorder or condition, i.e., arresting, reducing or delaying the development of the disease or a relapse thereof (in case of maintenance treatment) or at least one clinical or sub-clinical symptom thereof; and/or (3) relieving the disease, i.e., causing regression of the state, disorder or condition or at least one of its clinical or sub-clinical symptoms; and/or (4) causing a decrease in the severity of one or more symptoms of the disease.

As used herein, the term “in need of treatment” refers to a judgment made by a caregiver (e.g., physician, nurse, nurse practitioner, or individual in the case of humans; veterinarian in the case of animals, including non-human mammals), and such judgment is that a subject requires or will benefit from treatment. This judgment is made based on a variety of factors that are in the realm of a care giver's expertise, but that include the knowledge that the subject is ill, or will be ill, as the result of a condition that is treatable by the compounds of the present disclosure.

The term “administering” includes routes of administration which allow the agent of the disclosure to perform their intended function.

As used in the present invention, the term “pharmaceutical composition” refers to a mixture containing a therapeutic agent administered to an animal, for example a human, for treating or eliminating a particular disease or pathological condition that the animal suffers.

The term “effective amount” of an agent as provided herein refers to a sufficient amount of the ingredient to provide the desired regulation of a desired function. As will be pointed out below, the exact amount required will vary from subject to subject, depending on the disease state, physical conditions, age, sex, species and weight of the subject, the specific identity and formulation of the composition, etc. Dosage regimens may be adjusted to induce the optimum therapeutic response. For example, several divided doses may be administered daily or the dose may be proportionally reduced as indicated by the exigencies of the therapeutic situation. Thus, it is not possible to specify an exact “effective amount.” However, an appropriate effective amount can be determined by one of ordinary skill in the art using only routine experimentation.

The term “pharmaceutically acceptable” as used herein refers to compounds, materials, compositions, and/or dosage forms which are, within the scope of sound medical judgment, suitable for use in contact with the tissues of a subject (either a human or non-human animal) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. Suitable carriers, excipients, etc. can be found in standard pharmaceutical texts.

The term “wild type” refers to a nucleic acid or polypeptide in which the sequence is a form prevalent in a population, particularly humans of Asian descent. For purposes of this disclosure, a “wild type” PIAS1 refers to Homo sapiens protein inhibitor of activated STAT 1 (PIAS1), transcript variant 2, mRNANCBI Reference Sequence: NM_016166.3.

The term “a PIAS1 variant” when used in reference to PIAS1 polypeptide, refers to a polypeptide in which the sequence differs from the normal or wild-type sequence at a position that changes the amino acid sequence of the encoded polypeptide. For example, some variations or substitutions in the nucleotide sequence of PIAS1 alter a codon so that a different amino acid is encoded resulting in a variant polypeptide. Variant polypeptides can be located in the C-terminal region of PIAS1, such as S510G, A445T and T635M and one or more combinations thereof.

I. Identification of Genetic Modifiers of Nucleotide Repeat Disorders

Nucleotide repeat disorders generally show genetic anticipation: their age at onset and/or severity increases with each successive generation that inherits them. The number of repeats in the disease gene continues to increase as the disease gene is inherited. Longer repeat expansions are associated with genetic anticipation, earlier disease onset in successive generations, and earlier disease onset in general: however, the differences in age at the onset of these diseases are not all accounted for by repeat length, which implies the existence of additional modifying factors (Lesley Jones et al., DNA repair in the trinucleotide repeat disorders, The Lancet Neurology, Volume 16, ISSUE 1, P88-96, Jan. 1, 2017). For example, the CAG repeat lengths could only account for 50-70% of the variability in the polyQ diseases (HD, SCA2, SCA3).

Accordingly, the present disclosure proposes a novel method, which is essential for identifying genetic modifiers for such diseases. Particularly, the present disclosure provides a method of identifying a genetic modifier of a nucleotide repeat disorders, comprising: (a) providing the length of one or more nucleotide repeats in samples obtained from subjects to obtain nucleotide repeat load in genomes of the subjects; (b) clustering the subjects based on overall nucleotide repeat loads in the subject; (c) selecting from the subjects the late-onset subjects with higher nucleotide repeat load or the early-onset subjects with lower nucleotide repeat load; and (d) identifying one or more genetic modifiers delaying or promoting onset of a nucleotide repeat disorder.

The lengths of one or more nucleotide repeats in samples obtained from subjects are measured to obtain nucleotide repeat load. Then, the subjects are clustered based on the overall nucleotide repeat loads in the subject. In one embodiment, the clustering is performed using Euclidean distance and Ward's method. From the subjects, the late-onset subjects with higher nucleotide repeat load or the early-onset subjects with lower nucleotide repeat load are selected. In one embodiment, the nucleotide repeat load is a TNR load. In some embodiments, the TNR load is a CGG-repeat load, a CTG-repeat load, a GAA-repeat load or a CAG-repeat load.

Then, one or more genetic modifiers delaying or promoting onset of a nucleotide repeat disorder can thus be identified. Genetic modifiers, defined as genetic variants that can modify the phenotypic outcome of the primary disease-causing variant, are one such example. They can increase (known as an enhancer) or decrease (known as a suppressor) the severity of the disease condition. Modifier variants can change a target gene's phenotype by having a genetic, biochemical, or functional interaction with one or more target gene(s), or gene product(s). The degree of the effect of the modifiers can vary, which may result in large phenotypic variability and changes in penetrance.

In some embodiments of the disclosure, the present disclosure takes advantage of the overall “CAG-repeat load” from seven polyQ disease genes and cluster patients accordingly. In this way, two genetic modifiers are therefore separately identified in those early-onset patients with lower CAG-repeat loads as well as those late-onset patients with higher CAG-repeat loads by using a CAG repeat-related disease as a model system. To identify genetic modifiers (GMs) from patients who are most likely influenced by genetic modifiers, the repeat length information from the other (CAG)n-containing genes is collected, and the whole exome sequencing (WES) approach is used to include as many candidate genes as possible. It is found that less “CAG load”, less likely the age of onset (AO) influenced by these genes.

In a particular embodiment, the present disclosure aims to detect variants included in the group consisting of deletions, insertions and point changes such as variants affecting splice sites, missense mutation and nonsense mutations, preferably missense mutation and nonsense mutations. Typical techniques for detecting the presence of a variant may include restriction fragment length polymorphism, hybridization techniques, DNA sequencing, exonuclease resistance, microsequencing, solid phase extension using ddNTPs, extension in solution using ddNTPs, oligonucleotide ligation assays, methods for detecting single nucleotide polymorphisms such as dynamic allele-specific hybridization, ligation chain reaction, mini-sequencing, DNA “chips”, allele-specific oligonucleotide hybridization with single or dual-labelled probes merged with PCR or with molecular beacons, and others.

The present disclosure also provides computer systems that are programmed to implement methods of the disclosure. The present disclosure also provides a computer system for identifying a genetic modifier of a nucleotide repeat disorder, comprising:
a database that is configured to store data of the length of one or more nucleotide repeats in samples obtained from subjects to obtain nucleotide repeat load in genomes of the subjects; and
one or more computer processors operatively coupled to the database, wherein the one or more computer processors are individually or collectively programmed to cluster the subjects based on overall nucleotide repeat loads in the subject; select from the subjects the late-onset subjects with higher nucleotide repeat load or the early-onset subjects with lower nucleotide repeat load; and identify one or more genetic modifiers delaying or promoting onset of a nucleotide repeat disorder.

The present disclosure also provides a non-transitory computer-readable medium comprising machine-executable instructions which, upon execution by one or more computer processors, perform the method for identifying a genetic modifier of a nucleotide repeat disorder as described herein.

In one embodiment of the disclosure, the computer system can regulate various aspects of analysis, calculation, and identification of the present disclosure. The computer system can be an electronic device of a user or a computer system that is remotely located with respect to the electronic device. The electronic device can be a mobile electronic device.

The computer system includes a central processing unit (CPU, also “processor” and “computer processor” herein), which can be a single core or multi core processor, or a plurality of processors for parallel processing. The computer system also includes memory units (e.g., random-access memory, read-only memory, flash memory), electronic storage unit (e.g., hard disk and solid state disk), communication units (e.g., wired communication module and wireless communication module) for communicating with one or more other systems, and peripheral devices (e.g., memory units, data storage units and electronic display adapters). The memory units, data storage units, communication units and peripheral devices may be in communication with the CPU through a communication bus. The computer system can be operatively coupled to a computer network (“network”) with the aid of communication units. The network can be an Internet, or an internet and/or extranet, that is in communication with an internet. The network in some cases is a telecommunication and/or data network. In some embodiments, network can include a local area network (“LAN”), including without limitation an ethernet network; a Token-Ring network and/or the like; a wide-area network; a wireless wide area network (“WWAN”); a virtual network, such as a virtual private network (“VPN”); the Internet; an intranet; an extranet; a wireless network, including without limitation a network operating under any of the IEEE 802.11 suite of protocols, the Bluetooth™ protocol known in the art, and/or any other wireless protocol; and/or any combination of these and/or other networks. The network can include one or more computer servers, which can enable distributed computing, such as cloud computing. For example, one or more computer servers may enable cloud computing over the network (“the cloud”) to perform various aspects of analysis, calculation, and identification of the present disclosure. Such cloud computing may be provided by cloud computing platforms such as, for example, Amazon® Web Services (AWS), Microsoft® Azure, Google® Cloud Platform, and IBM® cloud. The network, in some cases with the aid of the computer system, can implement a peer-to-peer network, which may enable devices coupled to the computer system to behave as a client or a server.

The CPU can execute computer-readable instructions, stored on memory, which can be embodied in a program or software. The instructions are executed by the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC), microprocessor, core, or memory chip. It should be appreciated that the CPU can be any type of electronic circuitry.

The data storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the internet.

The computer system can communicate with one or more remote computer systems through the network. For instance, the computer system can communicate with a remote computer system of a user (e.g., a physician, a nurse, a caretaker, a patient, or a subject). Examples of remote computer systems include personal computers (e.g., portable PC), slate or tablet PC's (e.g., Apple® iPad, Samsung® Galaxy Tab), telephones, Smart phones (e.g., Apple® iPhone, Android®-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Methods and systems of the present disclosure can be implemented by way of one or more algorithms. An algorithm can be implemented by way of software upon execution by the central processing unit.

II. Agent and Method for Treating or Preventing Nucleotide Repeat Disorders

Huntington's disease (HD) is a dominantly inherited neurodegenerative disease with a typical onset of midlife. One of the most affected areas in the brain is the striatum, which plays an important role in coordinating body movements. The motor symptoms of HD include choreoathetosis and incoordination at early stages, followed by bradykinesia, rigidity and dyskinesia at later stages. Other clinical symptoms, such as cognitive decline and psychiatric aberrations, are also cardinal manifestations (Ross et al., The Lancet Neurology. 2011; 10(1):83-98). The disease-causing mutation is an expanded CAG repeat in exon 1 of the HTT gene (Soong et al., J Med Genet. 1995; 32(5):404-5). Mutant HTT (mHTT) usually undergoes proteolysis. The resultant N-terminal fragment of mHTT contains the expanded polyQ tract that is liable to form aggregates.

Accordingly, the present disclosure provides a method for treating or preventing a polyQ expansion disease in a subject in need of such treatment or prevention, comprising administering an effective amount of a PIAS1 variant or mutant or a recombinant nucleic acid molecule encoding the PIAS1 variant or mutant to the subject. Particularly, the present disclosure provides a method for treating or preventing early symptoms onset of the polyglutamine expansion disease in a subject in need of such treatment or prevention, comprising administering an effective amount of an agent for diminishing the effect of wild type PIAS1 in the subject

The present disclosure firstly shows that PIAS1 variants or mutant can modulate mutant Huntingtin or SCA3 and reduce their toxicity associated with HD or SCA3 by exhibiting a significantly lower ability to interact with mutant Huntingtin or SCA3. Particularly, the present disclosure found that the agent for diminishing the effect of wild type PIAS1, PIAS1 variant or mutant or a recombinant nucleic acid molecule encoding the PIAS1 variant or mutant is associated with early symptoms onset (i.e. age of onset, AO) in patients with SCA3 or HD. Structure and function of PIAS1 variants or mutant provide a potential base to design therapeutic treatments for HD or SCA3.

PIAS1 is a negative regulator of STAT1 and NF-kB in the interferon signaling pathway (Liu et al., Molecular and Cellular Biology. 2004; 25(3):1113-23). It is also an E3 SUMO ligase and contains a RING finger-like zinc-binding domain (RLD) (Eaton et al., The Journal Of Biological Chemistry. 2003; 278(35): 33416-21). In the SUMO modification pathway, SUMO proteins are first activated by an E1 SUMO-activating enzyme and then transferred to E2 SUMO conjugates. E3 SUMO ligases function as adaptors between E2 SUMO conjugation enzymes and target substrates to facilitate the SUMOylation reaction (Schmidt et al., Proc Natl Acad Sci. 2002; 99(5):2872-7; Tozluoglu et al., PLoS Computational Biology. 2010; 6(8)). An E3 SUMO ligase not only promotes the SUMOylation of a substrate but also controls substrate specificity (Rytinki et al., Cell Mol Life Sci 2009; 66:3029-41). SUMOylation is important in HD because both wild-type and mutant HTT (mHTT) can be modified by SUMO-1 or SUMO-2 at its N-terminus, which subsequently modulates the homeostasis of HTT proteins (O'Rourke et al., Cell Reports. 2013; 4:362-75). However, its association with clinical outcomes, such as disease onset or severity, for patients with HD has not been reported.

While not wishing to be limited by theory, it is believed that the age-at-onset (AO) of HD is inversely correlated with the length of CAG repeats of the HTT gene (Brinkman et al., The American Journal of Human Genetics. 1997; 60(5):1201-10). The longer the CAG repeat is, the faster the accumulation of toxic aggregates of mHTT in the neuronal nuclei, and hence, the AO is earlier (Genetic Modifiers of Huntington's Disease (GeM-HD) Consortium. CAG Repeat Not Polyglutamine Length Determines Timing of Huntington's Disease Onset. Cell. 2019; 178:887-900). Notably, the detrimental impact of CAG repeat length on the AO is only partial. Expression of genetic modifier(s) may accelerate or delay the AO of HD by modifying HD pathogenesis. More than 10 polyQ diseases with the expansion of CAG repeats in different host genes, have been identified, in addition to HD (Naphade et al., Neurotherapeutics 2019; 16(4):979-98). Among disease-causing genes, spinocerebellar ataxia type 3 (SCA3), which harbors a CAG repeat expansion in the ATXN3 gene, causes the most common polyQ disease in Taiwan (Soong et al., Arch Neurol. 2001; 58(7):1105-9).

By targeted sequencing of genes involved in proteostasis, PIAS1 is identified as a genetic modifier for a later-than-average AO of the disease. In some embodiments of the disclosure, the PIAS1 variants are N445T, S510G, and T635M, and the PIAS1 variants are located in the C-terminus of PIAS1. In one embodiment, the PIAS1 variant is the gene product of PIAS1 variant S510G. Furthermore, the agent for diminishing the effect of wild type PIAS1, PIAS1 variant or a recombinant nucleic acid molecule encoding the PIAS1 variant is provided in the disclosure for treating or preventing a polyQ expansion disease. The PIAS1 variant impairs its substrate interaction with HTT proteins and consequently reduces SUMOylation-mediated mHTT accumulation. While not wishing to be limited by theory, it is believed that since SUMOylation of mHTT at its N-terminus makes it more stable and thus easier to accumulate as inclusions (DeGuire et al., Journal of Biological Chemistry. 2018; 293(48):18540-58), the expression of PIAS1 variant changes the protein homeostasis of mHTT by increasing its turnover and consequently delays the disease onset.

In some embodiments of the disclosure, the wild-type Pias1 allele was replaced with the Pias1^{S510G} variant in animal models of HD, and the resultant HD/Pias1^{S510G/S510G} animals exhibited milder HD symptoms and fewer mHTT aggregates than those harboring wild-type Pias1. The HD/Pias1^{S510G/S510G} animals showed milder HD symptoms (in terms of body weight loss, muscle strength, motor balance, and life span) than HD/Pias1^{WT/WT} ones. The accumulation of mHTT aggregates in the striatum, a major hallmark of HD, was also lower in the HD/Pias1^{S501G/S510G} animals than in the HD/Pias1^{WT/WT} ones.

Spinocerebellar ataxia type 3, an inherited neurological disorder, is caused by expression of mutant ATXN3, which encodes a protein with a long stretch of polyQ that is aggregation-prone and detrimental to neurons. The present disclosure found the expression of mutant ATXN3 is regulated by PIAS1 at the protein level. PIAS1 enables the SUMOylation of ATXN3 and increases the accumulation of mutant ATXN3 in the insoluble fraction of protein lysates. The gene product of PIAS1 variant sustains its interaction with ATXN3; however, this variant exhibits a compromised activity in SUMO conjugation of mutant ATXN3, together with a decrease of protein aggregation and cell death. The findings thus provide a molecular basis to account for the identification and association of PIAS1 gene variant in late-onset patients.

Therefore, the agent for diminishing the effect of wild type PIAS1, PIAS1 variant or a recombinant nucleic acid molecule encoding the PIAS1 variant is for reducing accumulation of mutant polyQ proteins and/or for preventing mutant polyQ proteins aggregation and toxicity in the subject. Furthermore, the agent for diminishing the effect of wild type PIAS1, PIAS1 variant or a recombinant nucleic acid molecule encoding the PIAS1 variant is for lowering SUMOylation of mutant polyQ proteins, for de-stabilizing mutant polyQ proteins and/or for decrease of SUMO3-conjugation on mutant polyQ proteins in the subject.

PIAS1 stabilizes mutant polyQ proteins via SUMOylation and increases its accumulation in the insoluble fraction of protein lysates. The PIAS1 variant interacts proficiently with polyQ proteins, and it shows a marked decrease of SUMO3-conjugation on mutant polyQ proteins, along with a reduction of foci formation and cell death, and improvement of motor function.

In one embodiment, the PIAS1 variant or mutant prevents mutant polyQ proteins aggregation and toxicity by lowering its abundance in the insoluble fraction of protein lysates. The PIAS1 variant or mutant interacts ordinarily with polyQ proteins, but causes a decrease of SUMOylation on mutant polyQ proteins.

In one embodiment, the agent for diminishing the effect of wild type PIAS1 is an RNA interference agent (RNAi), a nucleic agent, an oligopepetide or a chemical agent. In some embodiments, the RNAi is a small inhibitory RNA (siRNA), a microRNA (miRNA), or a small hairpin RNA (shRNA).

RNA interference (RNAi) has been shown to be a useful tool for gene silencing in basic research of gene function and shows great promise as a therapeutic agent to suppress genes associated with the development of a number of diseases.

In one animal model of the present disclosure, RNAi is used to knock down the expression of wild type PIAS in SCA3 expressing ATXN3-84Q. It has been found that down-regulation of wild type PIAS improve various pathogenesis of SCA3, including degeneration, accumulation of pathogenic protein and motor function deficits of animals.

In certain aspects of any target gene silencing nucleic acid molecule described anywhere herein, the nucleic acid molecule is a RNA molecule. In certain aspects of any target gene silencing nucleic acid molecule described anywhere herein, the RNA molecule is a double stranded molecule (dsRNA), for example, for use in the RNA interference (RNAi) process. As used herein, a dsRNA molecule is a RNA molecule comprising at least one annealed, double stranded region. In certain aspects, the double stranded region comprises two separate RNA strands annealed together. In certain aspects, the double stranded region comprises one RNA strand annealed to itself, for example, as can be formed when a single RNA strand contains an inversely repeated sequences with a spacer in between. One of ordinary skill in the art will understand that complementary nucleic acid sequences are able to anneal to each other but that two sequences need not be 100% complementary to anneal. The amount of complementarity needed for annealing can be influenced by the annealing conditions such as temperature, pH, and ionic condition.

The present disclosure provides the PIAS1 variant or mutant, comprising one or more sequence changes located in the C-terminal region of PIAS1.

Certain aspects of this disclosure provide for a recombinant nucleic acid molecule, such as a DNA vector, comprising and/or encoding a nucleic acid molecule disclosed anywhere herein for silencing a target gene, including long dsRNA, hpRNA, and siRNA. Certain aspects provide for recombinant nucleic acid constructs comprising and/or encoding an RNAi precursor of a nucleic acid molecule disclosed anywhere herein for silencing a target gene, including long dsRNA, hpRNA, and siRNA.

Provided herein are host cells comprising, expressing, processing, and the like a dsRNA as described anywhere herein for inducing RNAi in an insect. In certain aspects, a host cell comprises a dsRNA molecule, siRNA molecule, a polynucleotide encoding a dsRNA molecule, and/or a construct or a dsRNA encoding segment thereof described anywhere herein. Representative examples of host cells include bacterial cells, fungal cells, yeast cells, plant cells and mammalian cells. One of ordinary skill will understand that there are many well-known methods for introducing a nucleic acid, such as a vector, into a host cell including well-known methods for generating transgenic cells. In certain aspects, the hose cell expresses a dsRNA molecule and/or produces siRNA to silence a target gene.

The following examples are provided to aid those skilled in the art in practicing the present disclosure.

### EXAMPLES

Methods

Patients. Age at onset (AO) was defined as the age (year) at the appearance of first symptoms-choreoathetosis in HD and gait unsteadiness in SCA3. Trinucleotide CAG repeats were analyzed in 127 symptomatic HD cases (female:male=76:51; AO: 42.61±12.36 years (11-73); CAG repeat length in the normal HTT alleles: 18.51±2.57 (12-29), CAG repeat length in the pathogenic HTT alleles: 46.18±5.37 (40-78)) and 210 symptomatic SCA3 cases (female:male=104:106; AO: 38.29±13.47 years (8-80); CAG repeat length in the normal ATXN3 alleles: 20.26±6.65 (14-36), CAG repeat length in the pathogenic ATXN3 alleles: 71.35±4.87 (55-87)) recruited from the Taipei Veterans General Hospital, Taipei. Informed consent was obtained from all included in the study (approval from the Institutional Review Board of Taipei Veterans General Hospital: “2015-11-010B”).

Cell culture and transfection. HeLa cells were cultured in DMEM (Gibco) with 10% FPS (Gibco) and incubated at 37° C. with 5% CO₂. ST14A cells (Rat striatal cells) were cultured in DMEM (HyClone) with 10% FPS (Gibco) and incubated at 33° C. with 5% CO₂. Cells were seeded on plates at 60-70% confluence, transfected with plasmid DNA by Lipofectamine 2000 at a ratio of 1:1 (HeLa cells) or 1:2 (ST14A cells) as per manufacturer's instructions.

Soluble insoluble protein fractionation. After rinse with PBS, cells were lysed in 1% Triton X-100 lysis buffer (50 nM Tris-HCl [pH 7.5], 150 nM NaCl, 1% NP-40, 1% sodium deoxycholate and 1% TritonX-100). Cell lysates were subjected to centrifugation (13000 rpm, 20 min) at 4° C. Supernatant was collected as a soluble fraction. The pellet was then re-suspended in lysis buffer containing 4% SDS and heated at 95° C. for 30 min as an insoluble fraction.

Cycloheximide chase assay. HeLa cells were transfected with pcDNA3.0, ATXN3-84Q and PIAS1 shRNA. After 20-hour transfection, HeLa cells were treated with 50 μg/mL cycloheximide (CHX). After CHX treatment, cells were harvested at 0, 2, 4, 8, 12 hours. Cells rinsed by PBS and lysed in a 0.2% SDS lysis buffer (50 mM Tris-HCl [pH 7.5], 150 mM NaCl, 1% NP-40, 1% sodium deoxycholate) supplemented with 1 mM Na₃VO₄, 1 mM DTT, 1 mM PMSF and protease inhibitor cocktail (Sigma). After centrifugation (13,000 rpm, 20 min, 4° C.) the supernatant was collected as protein lysate. The cell lysate was analyzed by Western blot by using anti-ATXN3 antibody.

Western blot analysis. Protein lysates were separated by SDS-PAGE and transferred to nitrocellulose membranes. The membranes were blocked with 5% non-fat milk in TBST (TBS with 0.1% Tween 20) at room temperature for 1 hour, probed with primary antibodies at 4° C. overnight, rinsed with TBST three times (10 min, each), and probed with secondary antibodies at room temperature. After washing three times with TBST, the membranes were incubated with ECL Plus Western Blotting Detection Reagents (Amerham) and monitored by Luminescence Imaging System (Fuji LAS-4000). The signals were quantified by Multi Gauge.

Trypan blue assay. 48-hour post transfection, suspended ST14A cells were collected from medium by centrifugation (2000 rpm, 5 min) and attached cells were removed from plates by trypsinization. Collected cells were pooled and mixed with trypan blue dye. After 3 min incubation, the mixture was dropped on hemocytometer and counted the numbers of live and dead cells under microscope.

Immunoprecipitation. Cells were rinsed by PBS and lysed in a lysis buffer (50 nM Tris-HCl [pH 7.5], 150 nM NaCl, 1% NP-40, 1% sodium deoxycholate) supplemented with 1 mM Na₃VO₄, 1 mM DTT, 1 mM PMSF and protease inhibitor cocktail (Sigma). After centrifugation (13000 rpm, 20 min) at 4° C., the supernatant was collected as a protein lysate. Following pre-cleaning of protein lysate (1 μg) with protein G agarose beads, the supernatant was removed and incubated with antibody at 4° C. overnight. The mixture was co-incubated with new protein G agarose beads at 4° C. for 1 hour. Immuno-precipitate was then washed three times using IP buffer (lysis buffer:PBS=1:2), mixed with sample buffer, and analyzed by Western blot.

In vivo SUMOylation assay. Cells were rinsed by PBS containing 20 mM NEM (N-Ethylmaleimide) and lysed in lysis buffer supplemented with 1 mM Na₃VO₄, 1 mM DTT, 1 mM PMSF, protease inhibitor cocktail (Sigma), and 20 mM NEM. After centrifugation (13000 rpm, 20 min) at 4° C., supernatant was collected and subjected to immunoprecipitation (IP) as described above, except with the supplementation of NEM in each step of procedures.

In vitro transcription translation and SUMOylation assay. 21 DNA template (pSG5-ATXN3-28Q or pSG5-ATXN3-84Q) were incubated with 40 μl master mix TNT® Quick Coupled Reticulocyte Lysate (Promega—L1170) for 2 hr at 30° C., produced ATXN3 were collected by immunoprecipitation (IP). ATXN3 protein captured on beads were subjected to in vitro SUMOylation assay kit (Abcam-ab139470). Followed the manual, ATXN3 were incubated with SUMO3 protein, SUMO Activating Enzyme E1, Ubc9 (SUMO E2), Mg-ATP Solution and purified wild-type or variant 3 PIAS1 in SUMOylation Buffer for 1 hr at 37° C. After the reaction, beads were washed three times using PBS containing 20 mM NEM (N-Ethylmaleimide) and added with 2×SDS sample buffer for analysis by Western blot.

GST pull-down assay. 5 μg of bacterially purified GST-Ubc9 fusion protein and GST protein were incubated with 15 μl glutathione sepharose beads (Glutathione Sepharose® 4B, Merck) in 300 μl binding buffer (10 mM HEPES pH7.5, 0.5 mM EDTA, 0.1% NP-40, 50 mM NaCl, 0.5 mM DTT) for 1 hr at 4° C. The samples were then washed and blocked in buffer containing 5 mg/ml of BSA for 1 hr. GST-fused protein were incubated with PIAS1 wild-type or variant 3 (5 g) and ATXN3 protein in 300 μl binding buffer for 1.5 hr. The sample were washed with high-salted washing buffer (binding buffer containing 100 mM NaCl) four times and added with 2×SDS sample buffer for further analysis. Sample were examined by Coomassie Blue staining and Western blot with anti-PIAS1 antibody.

Targeted Gene Sequencing and Bioinformatics Analysis. A targeted sequencing panel covering the coding regions of 583 genes of six protein homeostasis pathways and the mTOR signaling pathway was designed using NimbleDesign Software (Roche NimbleGen, Madison, Wis., USA). Targeted regions were enriched with the NimbleGen SeqCap EZ Choice Library system. The enriched samples were sequenced on an Illumina HiSeq 2500 platform (Illumina, San Diego, Calif., USA) for 100-bp paired-end sequencing. Raw reads were aligned to the human reference genome GRCh38 and processed following GATK Best Practices (McKenna et al., Genome Research. 2010; 20(9):1297-303; DePristo et al., Nature Genetics. 2011; 43:491-8). Variants were called with the GATK HaplotypeCaller software in GVCF mode and annotated with the Ensembl Variant Effect Predictor tool (McLaren et al., Genome Biology. 2016; 17(1):122).

Plasmid Construction. The DNA fragment of human PIAS1 was amplified from pGEMT-PIAS1 (Sino Biological US Inc., PA, USA) by polymerase chain reaction (PCR) using specific primers and subcloned into a pcDNA3.1/V5-His-TOPO vector (Invitrogen, Carlsbad, Calif., USA). Mutations of PIAS1^{A445T}, PIAS1^{T635M}, PIAS1^{S510G}, PIAS1^{S510A} and PIAS1^{S510D} were generated from pcDNA3.1-PIAS1^{WT} by standard site-directed mutagenesis methods using specific primers and subcloned into a pcDNA3.1/V5-His-TOPO vector (Invitrogen, Carlsbad, Calif., USA). The His-tagged S/T rich region of PIAS1 was amplified from pcDNA3.1-PIAS1^{WT} by PCR using specific primers and subcloned into a pRSETA-6×His vector (Invitrogen, Carlsbad, Calif., USA) using a TOOLS UltraFast PCR cloning kit.

Recombinant Protein Purification. BL21 cells were transformed with pGEX-4T-Q25-HTTex1, pGEX-4T-Q43-HTTex1, 6×His-PIAS1^{WT}, 6×His-PIAS1^{S510G} or 6×His-PIAS1-ST rich region only for the production of recombinant proteins. Expression of recombinant proteins was induced by isopropyl p-D-1-thiogalactopyranoside (IPTG, 1 mM) at 25° C. overnight for GST-tagged proteins and 37° C. for 2 hrs for His-tagged proteins using standard protocols. For the production of Q25-HTTexi and Q43-HTTexi, bacterial pellets were resuspended in lysis buffer (50 mM sodium phosphate, 200 mM NaCl, 0.1 mM phenylmethanesulfonyl fluoride and 1% glycerol, pH 8) and sonicated. The lysates were centrifuged (6000 Xg, 20 min, 4° C.) to harvest the supernatant, which was mixed with glutathione beads (Sigma-Aldrich, St. Louis, Mo., USA) at 4° C. for 2 hrs and eluted with elution buffer (10 mM glutathione and 500 mM Tris, pH 8). For the production of His-tagged recombinant proteins, culture pellets were resuspended in lysis buffer (50 mM NaH₂PO₄, 500 mM NaCl, 10 mM imidazole, 0.1 mM phenylmethanesulfonyl fluoride and 1% glycerol, pH 8) and sonicated. After centrifugation to remove cellular debris (13000 Xg, 15 min, 4° C.), the supernatant was harvested and mixed with Ni-NTA beads (Sigma-Aldrich, St. Louis, Mo., USA) at 4° C. for 1 hr and eluted with elution buffer (250 mM imidazole in 50 mM NaH₂PO₄ and 500 mM NaCl, pH 8). The purified recombinant proteins were further dialyzed overnight in PBS (100 mM Na2HPO4, 18 mM KH2PO4, 137 mM NaCl and 27 mM KCl, pH 7.4) containing 1% glycerol at 4° C.

Cell Culture and Transfection. HEK293T cells were cultured in Dulbecco's modified Eagle's medium (DMEM; Invitrogen, Carlsbad, Calif., USA) supplemented with 10% heat-inactivated fetal bovine serum (FBS). The cells were incubated in a humidified incubator at 37° C. with 10% CO₂. Transfection was performed using T-Pro NTR II (Ji-Feng Biotechnology, Taiwan) following the manufacturer's protocol.

GST Pull-down Assay. Cell lysates were harvested using a nondenaturing lysis buffer (137 mM NaCl, 1% NP-40, 20 mM Tris pH 8.0, and 1 mM EDTA) containing a protease inhibitor cocktail and 1× phosphatase inhibitor cocktail (Roche) and subjected to rotation at 4° C. for 1 hr. Purified recombinant GST and GST-Q25-HTT_{EX1} or GST-Q43-HTT_{EX1} fusion proteins (20 pg) were incubated with 50 pL of glutathione beads at 4° C. for 1 hr on a rolling wheel. Cell lysates (1 mg) containing PIAS1^{WT} or PIAS1^{S510G} were added to the GST fusion protein and incubated at 4° C. for 1 hr on a rolling wheel to allow complex formation. The bound proteins were analyzed with SDS-PAGE and Western blotting. For the protein interaction analysis, purified recombinant GST (5 pg) or GST-Q25-HTT_{EX1} fusion proteins (5 pg) were incubated with 20 pL of glutathione beads in 300 pL of binding buffer (10 mM HEPES pH 7.5, 0.5 M EDTA, 0.1% NP-40, 50 mM NaCl, and 0.5 mM DTT) at 4° C. for 1 hr on a rolling wheel. Then, the cells were blocked with 1% BSA at 4° C. for 1 hr on a rolling wheel. The purified recombinant 6×His-PIAS1-S/T-rich region (5 pg) was added to the GST fusion protein and incubated at 4° C. for 1 hr on a rolling wheel to allow complex formation. The complexes were then washed with highly salted washing buffer (10 mM HEPES pH 7.5, 0.5 M EDTA, 0.1% NP-40, 100 mM NaCl, and 0.5 mM DTT). The bound proteins were analyzed with SDS-PAGE and Western blotting.

SDS-PAGE and Western BlotAnalysis. Cells or brain tissues were lysed with RIPA lysis buffer (150 mM sodium chloride, Triton-X 100, 0.5% sodium deoxycholate, 0.1% SDS, and 50 mM Tris-HCl, pH 8.0) containing 1× protease inhibitor cocktail and 1× phosphatase inhibitor cocktail to prepare total lysates, which were rotated at 4° C. for 1 hr. Protein concentrations were determined with a Bio-Rad protein assay kit (Bio-Rad, Hercules, Calif., USA). Protein samples were separated on 8-10% SDS-PAGE gels and electrophoretically transferred onto PVDF membranes (Millipore, Billerica, Mass., USA). The membranes were incubated overnight at 4° C. with the following primary antibodies: anti-PIAS1 (Abcam; ab109388 and ab32219), anti-SUMO-2/3 (Cell Signaling; 18H8 and Abcam; ab3742), anti-actin (Sigma; A2066), anti-GAPDH (GeneTex; GTX627408), anti-GST (Abcam; ab92), anti-HA (Sigma-Aldrich; 11867423001) or anti-HTT (Habel; a custom antibody raised against protein translated from the exon 1 of HTT (designated HTT^{ex1}) and favors detection of oligomeric HTT^{ex1}. Immunoreactive bands were detected by enhanced chemiluminescence (ECL; Millipore) and recorded on Kodak XAR-5 film (Rochester, N.Y., USA) or UVP ChemiDoc-It Imaging System (Upland, Calif., USA).

In Vitro SUMOylation Assay. Purified 6×His-PIAS1^{WT} or 6×His-PIAS1^{S510G} (3.0 pg) was incubated with 0.2 pg of GST-SAE1/2 (E1), 2 pg of His-UBC9 (E2), 4 pg of His-SUMO-2, and 1 pg of GST-Q43HTT_{EX1} at 37° C. for 60 min in 40 pl of SUMOylation reaction buffer containing 2 mM ATP, 20 mM HEPES (pH 7.5) and 5 mM MgCb (Chang et al., Molecular Cell. 2011; 42(1):62-74). After incubation, the proteins were subjected to SDS-PAGE followed by Western blot analyses.

Filter Trap Assay. Cell lysates were harvested using RIPA lysis buffer (150 mM sodium chloride, Triton-X 100, 0.5% sodium deoxycholate, 0.1% SDS, and 50 mM Tris-HCl, pH 8.0) containing 1× protease inhibitor cocktail and 1× phosphatase inhibitor cocktail and subjected to rotation at 4° C. for 1 hr. Protein concentrations were determined with a Bio-Rad protein assay kit. Protein samples were prepared in PBS containing 2% SDS. The samples were loaded onto a slot blot manifold (Bio-Rad, Hercules, Calif., USA) with a cellulose acetate membrane (0.2-pm pore size) and washed with PBS containing 2% SDS. The blots were analyzed with an anti-HTT antibody (Habe 1). Immunoreactive bands were detected by enhanced chemiluminescence and recorded using Kodak XAR-5 film.

Animals. R6/2 (B6CBA-Tg(HDexon1)62 Gpb/1J) mice were purchased from the Jackson Laboratory (Bar Harbor, Me., USA) (36) and maintained in the animal facility of the Institute of Biomedical Science (IBMS) at Academia Sinica (Taipei, Taiwan) under standard conditions. The genotype of the offspring was verified by amplification of the human mHTT gene from genomic DNA isolated from mouse tails using the following primers: 5′-CCGCTCAGGTTCTGCTTTTA-3′ (SEQ ID NO: 1), 5′-GGCTGAG-GAAGCTGAGGAG-3′ (SEQ ID NO: 2). Pias1^{S510G} mice were generated using the CRISPR/Cas9 technique by the Transgenic Core Facility at Academia Sinica, Taiwan (AS-CFII-108-104). Specific nucleotide editing of the mouse Piasl gene was carried out at genomic position 62971842 (exon 12 of mouse Piasl) using standard CRISPR/Cas9 techniques. The following primers were used in the genome typing of the Pias1^{S510G} mice: 5′-GTAGGACTTATTGTGGTGTATACAATTGCATTTG-3′ (the forward primer) (SEQ ID NO: 3) and 5′-CAGTATTT CCAGAGCAGTGAGCGC-3′ (the reverse primer) (SEQ ID NO: 4). Because the knock-in of S510G created an additional Haelll site, the resultant DNA fragments were treated with Haelll at 37° C. for 1 hr to differentiate the wild-type and knock-in animals. The resultant Pias1^{S510G/S510G} mice were crossed with R6/2 mice to generate R6 2-Pias1^{S510/S510} mice. These animals were housed with a 12-hr light/12-hr dark cycle. All animal experimental procedures were performed in accordance with the guidelines established by the Institutional Animal Care and Use Committee (IACUC) of the IBMS at Academia Sinica.

Behavioral Analysis. The body weights of the mice were recorded for mice 7 to 14 weeks old. For grip strength, force was applied to the mouse by pulling its tail. When the animal released its grip, the maximum force was measured using a Grip Strength-Meter (TSE Systems, Inc., MO, USA). For clasping assessment, mice were suspended by the tail for 30 sec to score the clasping of limbs, as previously described (Hsu et al., Movement Disorder. 2019; 34(6):845-57). For the vertical pole test, mice were placed facing downward on a vertical pole. The total time to descend was recorded.

Immunofluorescence staining and image analyses. Mice were transcardially perfused with saline, postfixed in 4% paraformaldehyde for 3 days and equilibrated in 30% sucrose for 2 days at 4° C. Brain sections (20 pm) were incubated with a primary antibody at 4° C. in a humidified chamber for 1-2 days. The primary antibodies used included anti-HTT antibody (Habe 1) and anti-SUMO-2/3 (Abcam; ab3742). Images were captured using a Zeiss LSM 700 Stage confocal microscope with Zen 2012 software (Carl Zeiss, Germany). For each genotype group, six brains were analyzed for all the experiments. For quantitation, three random images were captured of each brain. For each striatal section, ten equally spaced frames throughout the striatal section were captured, and stack images were obtained for each brain using confocal microscopy at comparable sections.

Images were then analyzed by MetaMorph Microscopy Automation & Image Analysis software (Molecular Devices, USA) using the multiwavelength cell scoring application. Nonbiased analysis of the images was performed by using a fully automated journal script.

In Situ Proximity Ligation Assay (PLA). PLA was performed according to the manufacturer's protocol and as described previously (DiFiglia et al., Neuron. 1995; 14:1075-81, Ristic et al., Proteostasis: Methods and Protocols. 2016:279-90). Brain sections (20 pm) were incubated with anti-SUMO-2/3 (Abcam; ab3742) and anti-HTT antibodies at 4° C. in a humidified chamber for 1-2 days, followed by incubation with Duolink PLA probes (Sigma-Aldrich, St. Louis, Mo., USA) and measurement with a Duolink detection reagent kit (Sigma-Aldrich, St. Louis, Mo., USA). For each genotype group, six brains were analyzed for all the experiments. For quantitation, three random images were captured of each brain. For each striatal section, seven equally spaced frames throughout the striatal section were captured, and stack images were obtained for each brain using confocal microscopy at comparable sections. Images were captured using a Zeiss LSM 700 Stage confocal microscope with Zen 2012 software (Carl Zeiss, Germany) and analyzed with MetaMorph Microscopy Automation & Image Analysis software (Molecular Devices, USA) using the multiwavelength cell scoring application. Nonbiased analysis of the images was performed by using a fully automated journal script.

Statistical analysis. The data were analyzed using GraphPad Prism 6 (GraphPad Software, San Diego, Calif., USA) software. The data are presented as the means S.E.M. Statistical significance was determined by Student's t test, one-way ANOVA or two-way ANOVA as indicated. p-values<0.05 were considered statistically significant.

### Example 1 Identification of Genetic Modifiers of Nucleotide Repeat Disorders

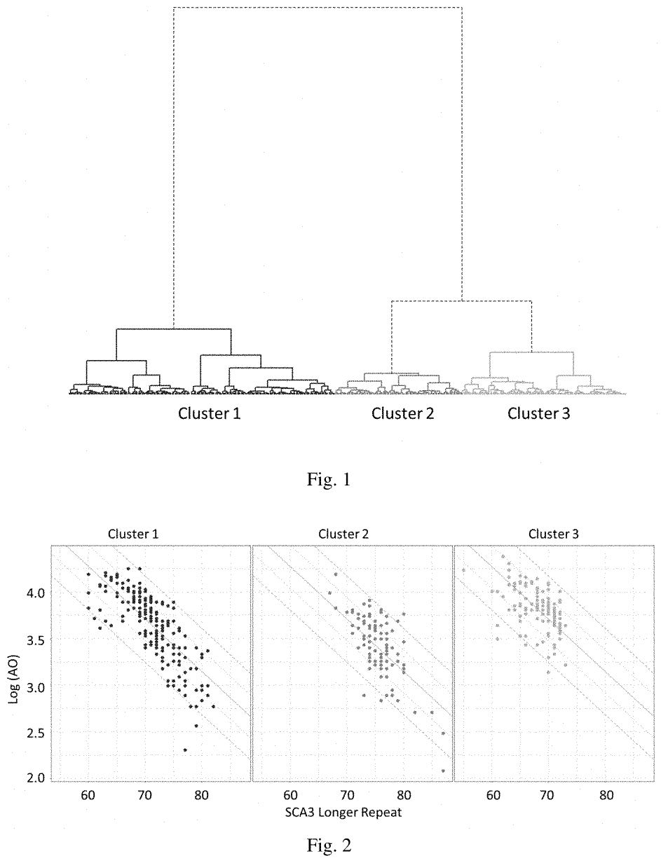

To identify the genetic modifiers of polyQ diseases, we measure the CAG repeats of seven polyQ disease causing genes for each polyQ disease patient first. These genes are: ATXN1, ATXN2, ATXN3, CACNA1A, TBP, ATN1 and HTT, which are the disease-causing genes for SCA1, SCA2, SCA3, SCA6, SCA17, DRPLA and HD, respectively. While the numbers of CAG repeat in both alleles of each disease-causing gene are available, we perform a clustering analysis with Euclidean distance and Ward's method for a cohort of 361 SCA3 patients based on these repeat numbers. The resultant dendrogram is shown as FIG. 1.

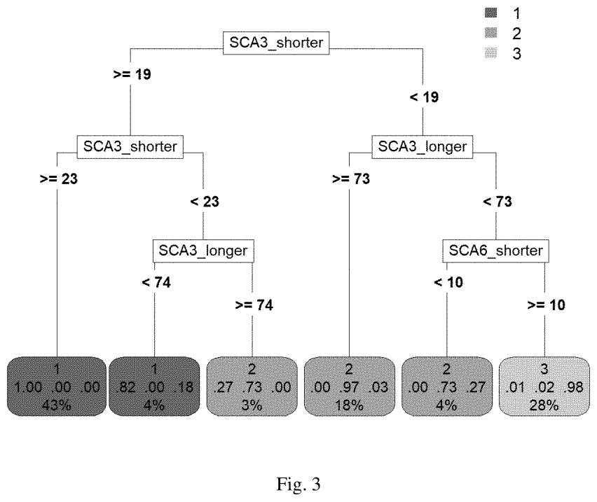

As shown in FIG. 1, there are three main clusters, of which two were separated from the same branch. While we illustrate the data as a regular scatter plot (FIG. 2), where x-axis represents the numbers of CAG repeat in patients' disease alleles and y-axis corresponds to the natural logarithm of patients' ages at onset, there is no significant difference in distribution between these three clusters. By using an intuitive and interpretable decision tree algorithm, we can identify discriminating features to distinguish these three clusters with simple rules (FIG. 3). We define the CAG load of a patient as the overall repeat numbers in the seven polyQ disease causing genes. While we observe the CAG loads distribution of patients of different clusters, we find that the mean CAG load of cluster 1 is higher than the mean values of the other two clusters. The CAG load distributions of cluster 2 and 3 are closer but distinguishable. In this view, the genetic backgrounds of patients of different clusters are indeed different.

The patients with higher CAG loads behave higher cellular burden. As a result, they would tend to have earlier age of onset time than average, i.e., earlier than average (ETA) patients. Based on this scenario, if we can identify a group of later than average (LTA) patients of higher CAG loads, we may have higher chances to identify genetic modifiers which can postpone the disease onset time. On the other hand, if we focus on the ETA patients with lower CAG loads, we may have higher chances to identify genetic modifiers which can facilitate the disease onset time.

### Example 2 Effect of PIAS1 Variants on ATXN3

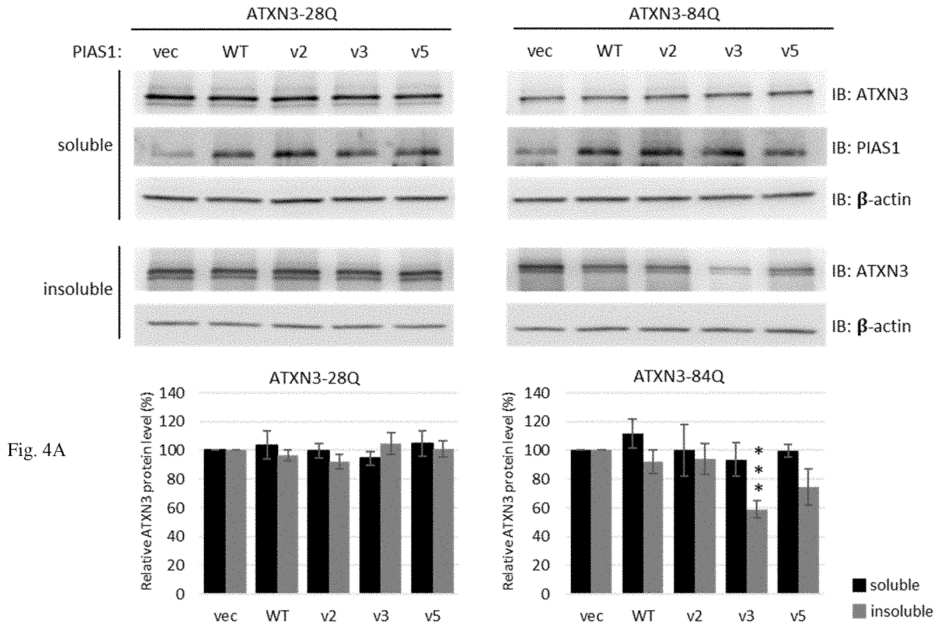

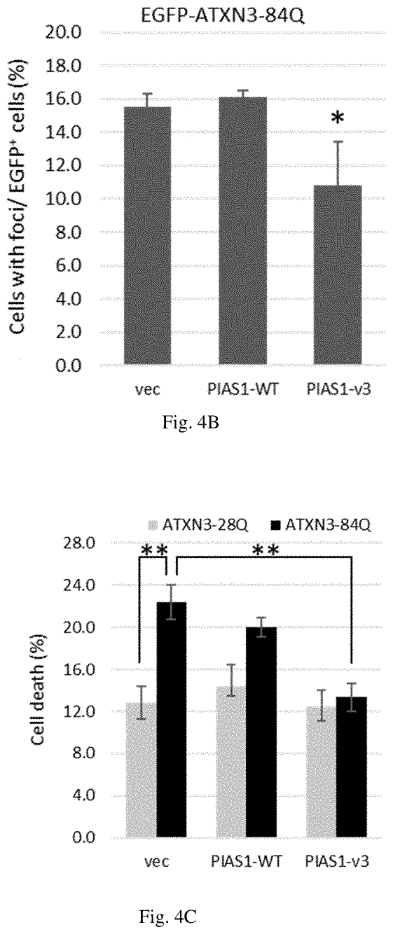

SCA3 is attributed by the expression of mutant ATXA3, which encodes a protein containing a long stretch of polyQ track that is aggregation-prone and detrimental to cells. In order to characterize the effect of PIAS1 gene variants on ATXN3, cells expressing either normal ATXN3 (ATXN3-28Q) or mutant ATXN3 (ATXN3-84Q) were introduced with wild-type PIAS1 and PIAS1 gene variants. We found that PIAS1 gene variant 3 (Pias1^{S510G}, v3) does not affect ATX3 gene expression at RNA level, but it causes a substantial reduction of mutant ATXN3 in the insoluble fraction of protein lysates (FIG. 4 (A)). Typically, insoluble fraction of mutant ATXN3 has a propensity for protein aggregation or foci formation that is harmful to cells. When PIAS1 gene variant 3 was introduced into murine neuronal cells expressing EGFP-ATXN3-84Q, it resulted in a decrease of foci formation as compared to cells expressing wild-type PIAS1 (FIG. 4 (B)). Accordingly, the elevated cell lethality caused by mutant ATXN3 was reversed by this gene variant (FIG. 4 (C)). Our results thus suggest that PIAS1 gene variant 3 has a nature of preventing mutant ATXN3 aggregation and toxicity by lowering its abundance in the insoluble fraction of protein lysates.

### Example 3 PIAS1 Knockdown Reduces ATXN3 Level

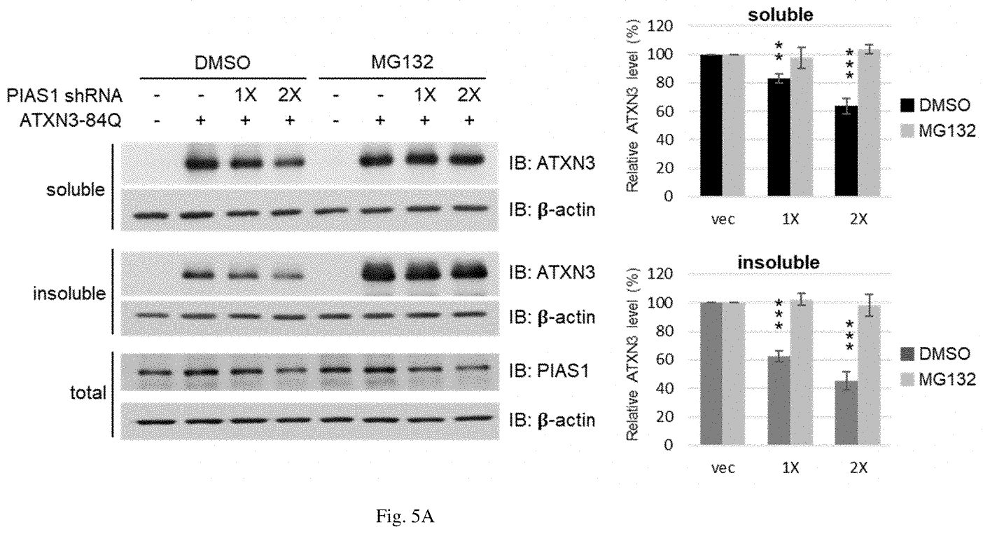

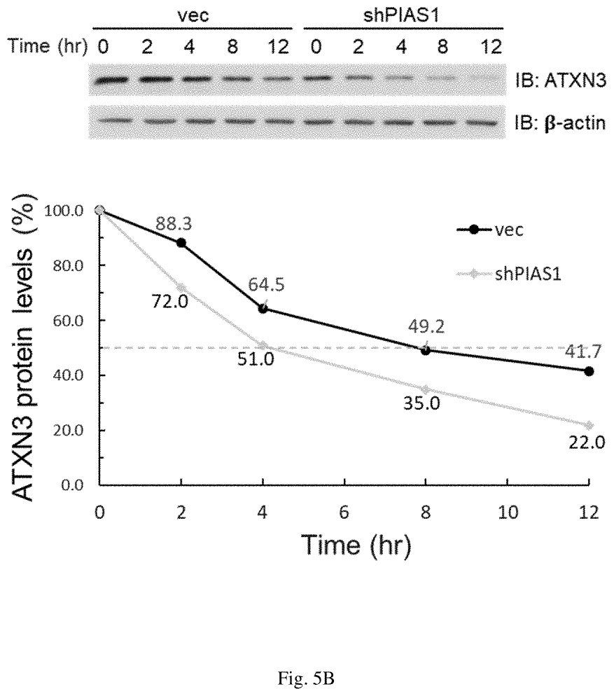

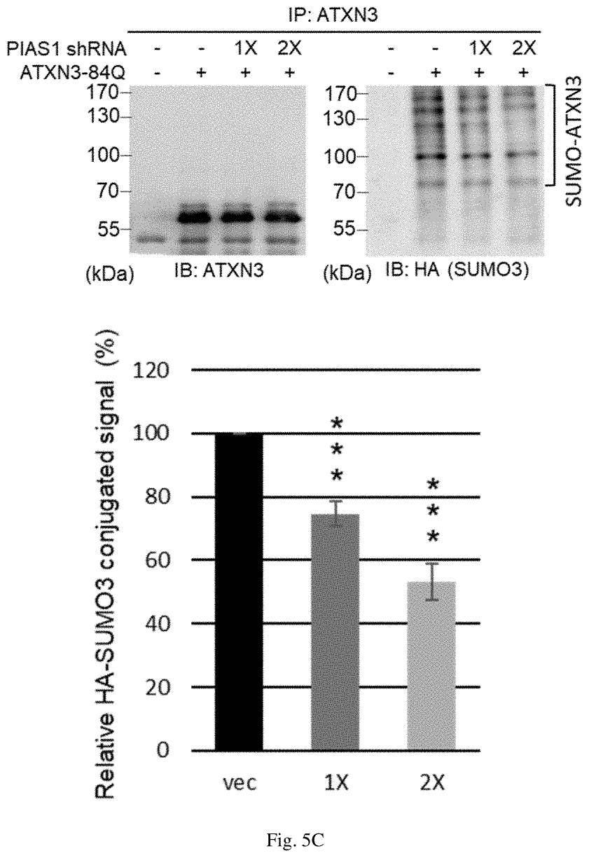

It is not known whether PIAS1 has a regulatory role in ATXN3. To this end, cells expressing ATXN3 were subjected to PIAS1 shRNA knockdown by different doses of shRNA, and analyzed the protein level of ATXN3. Our results showed that the mutant ATXN3 protein levels were reduced in a dose dependent manner when PIAS1 knockdown, especially the ATXN3 proteins in insoluble fraction (FIG. 5 (A)). Besides, MG132 prevents the reduction of ATXN3-84Q caused by PIAS1 knockdown and the mutant protein in the insoluble fraction is most stabilized by MG132. This imply that PIAS1 can stable ATXN3 proteins and prevent ATXN3 degradation by proteasome. In line with this notion, ATXN3 protein half-life reduced due to the protein stability reduced when PIAS1 knockdown (FIG. 5 (B)). PIAS1, a SUMO E3 ligase, regulates a variety of proteins through its SUMOylation activity. While Huntington's disease protein has been identified as a substrate of PIAS1, it is not known whether PIAS1 has a regulatory role in mutant ATXN3 via SUMO modification. Accordingly, cells expressing HA-tagged SUMO3 and ATXN3-84Q were subjected to PIAS1 shRNA knockdown and analyzed the level of SUMO-conjugated ATXN3-84Q. We found that SUMO3 enables to conjugate with mutant ATXN3; however, the quantity of ATXN3 with such conjugation is decreased by PIAS1 knockdown in a dose dependent fashion (FIG. 5 (C)). Similarly, the protein accumulation and SUMO conjugation of ATXN3 with 28Q were regulated by PIAS1. And not surprisingly, PIAS1 knockdown were not influence ATXN3 mRNA level. SUMOylation, in general, protects proteins from proteasome-mediated degradation, the amount of ATXN3 was further assessed in samples treated with MG132, a proteasome inhibitor. Our results showed that MG132 prevents the reduction of ATXN3 caused by PIAS1 knockdown. In addition, the mutant protein in the insoluble fraction is most stabilized by MG132, suggesting the species of ATXN3 that eventually accumulates in the insoluble fraction of protein lysates is more susceptible to the regulation of PIAS1 via SUMO3 conjugation.

### Example 4 PIAS1 Gene Variant 3 is Associated with Late-Onset Patients

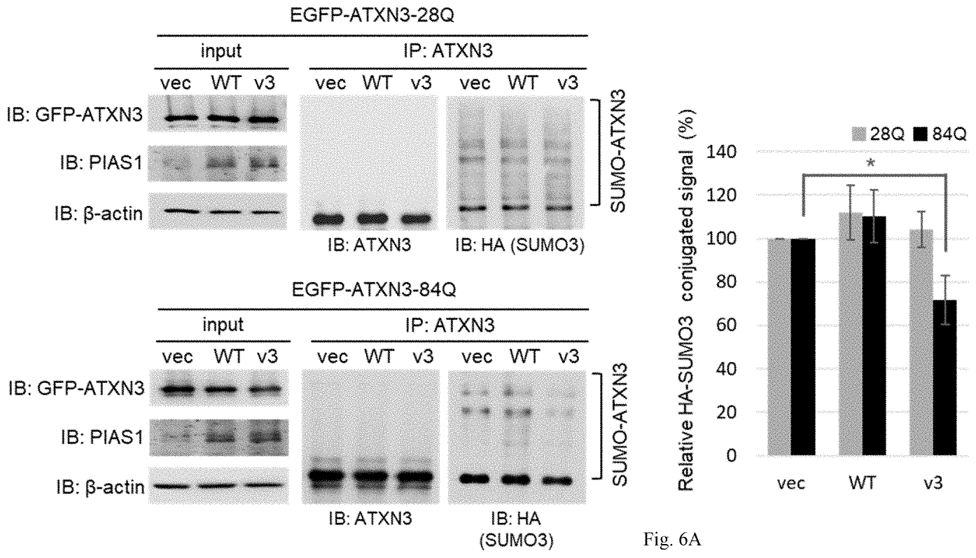

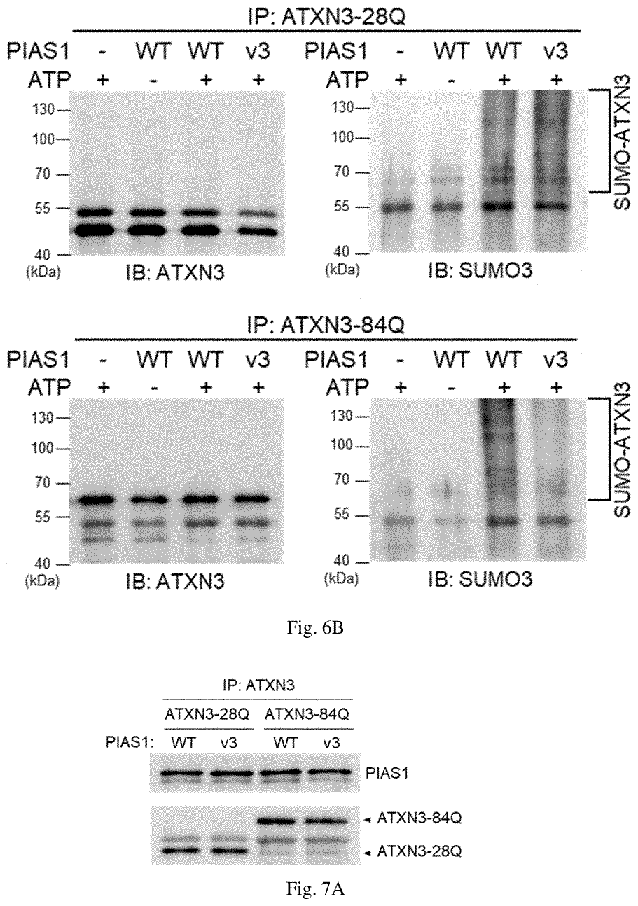

To further evaluate the effect of PIAS1 gene variant 3 on wild-type and mutant ATXN3, Cells expressing either EGFP-ATXN3-28Q or EGFP-ATXN3-84Q were subjected to in vivo SUMOylation assay. We found that the conjugation of SUMO3 to ATXN3-84Q is compromised by PIAS1 gene variant 3 (FIG. 6 (A)), which provide a molecular basis to account for the consequence of mutant ATXN3 reduction in the insoluble fraction of protein lysates (FIG. 6 (A)). Unexpected, only the mutant ATXN3 were affected by PIAS1 gene variant 3, the SUMO conjugation of normal length ATXN3 (EGFP-ATXN3-28Q) did not decrease when PIAS1 variant 3 expression. It means that PIAS1 gene variant 3 selectivity influence of mutant ATXN3. To date, it is unknown any protein can function as ATXN3 SUMO E3-ligase. These results demonstrate that PIAS1 is highly related with ATXN3 SUMO conjugation. Furthermore, we used recombinant protein system to verify PIAS1 gene variant 3 is direct or indirect effect. When PIAS1 and ATP (ATP is necessary for SUMOylation activation) participated the reaction process, formed ATXN3 SUMO conjugation (FIG. 6 (B)), both normal and mutant ATXN3. Therefore, PIAS1 is SUMO E3 ligase of ATXN3. In line with expectations, PIAS1 gene variant 3 has compromised activity of SUMO conjugation on mutant ATXN3 without normal ATXN3 affected.

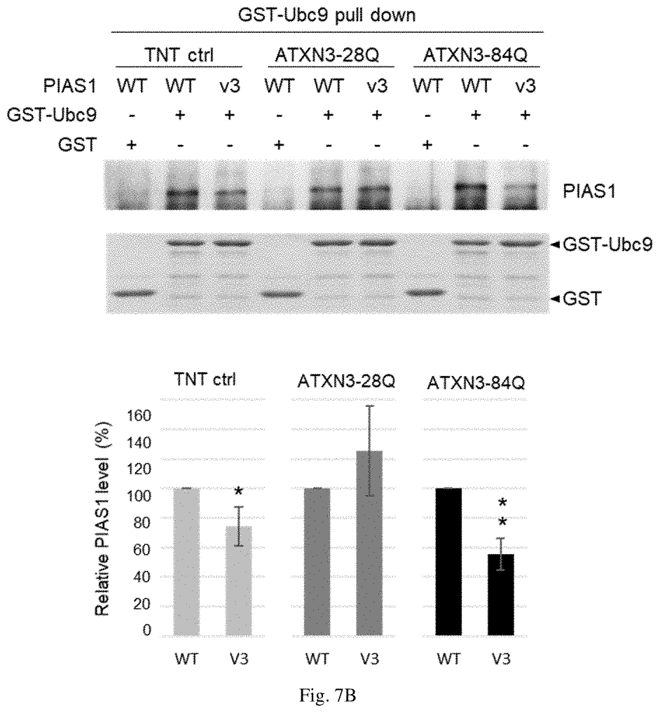

This signified that PIAS1 gene variant 3 is very unique. To further understanding this unique property, we focused on the mechanism of SUMOylation. PIAS1 served as SUMO E3-ligase, it interacts with substrates and transfers SUMO proteins. We found PIAS1 can interact with both normal and mutant ATXN3 (FIG. 7 (A)). However, the protein-protein interaction between PIAS1 and ATXN3 is not affected by the gene variant, suggesting the loss of SUMO3 conjugation by PIAS1 gene variant 3 is likely resulting from a compromised E3 ligase activity, rather than by a defect of substrate recognition. This results also can be found in cell system. In addition, PIAS1 interacts with ATXN3 by PINIT and RING domains that are far away from the site of variant 3, corresponding with the results that PIAS1 gene variant 3 does not affect the interaction between PIAS1 and ATXN3. Apart from PIAS1 interacts with substrate, interacting with SUMO-Conjugating Enzyme E2 UBC9 is also a key of SUMOylation. We used GST pulled down assay to verify interaction between PIAS1 and UBC9. Our results show that PIAS1 can interact with UBC9, and PIAS1 gene variant 3 has compromised ability of interacting with UBC9 (FIG. 7 (B)). More attractively, PIAS1 interact ordinarily with UBC9 when ATXN3-28Q were added in; however, interaction between PIAS1 and UBC9 were weaker with ATXN3-84Q. It reveals that substrate may influence PIAS1's ability to interact with UBC9 and explain the feature that PIAS1 selectivity affects mutant ATXN3.

Our data, for the first time, demonstrated that ATXN3 is under the regulation of PIAS1. In particular, PIAS1 is ATXN3 SUMO E3-ligase and can stabilize mutant ATXN3 via SUMOylation and increases its accumulation in the insoluble fraction of protein lysates, which could lead to an elevated protein aggregation and cell lethality. While the gene product of PIAS1 gene variant 3 interacts proficiently with ATXN3, but is deficient in interacting with UBC9 when mutant ATXN3 participated. It shows a marked decrease of SUMO3-conjugation on mutant ATXN3, along with a reduction of foci formation and cell death. Our findings provide a detail of molecular mechanism to account for the clinical observations, in which PIAS1 gene variant 3 is associated with late-onset patients.

### Example 5 SCA3 Fly Model

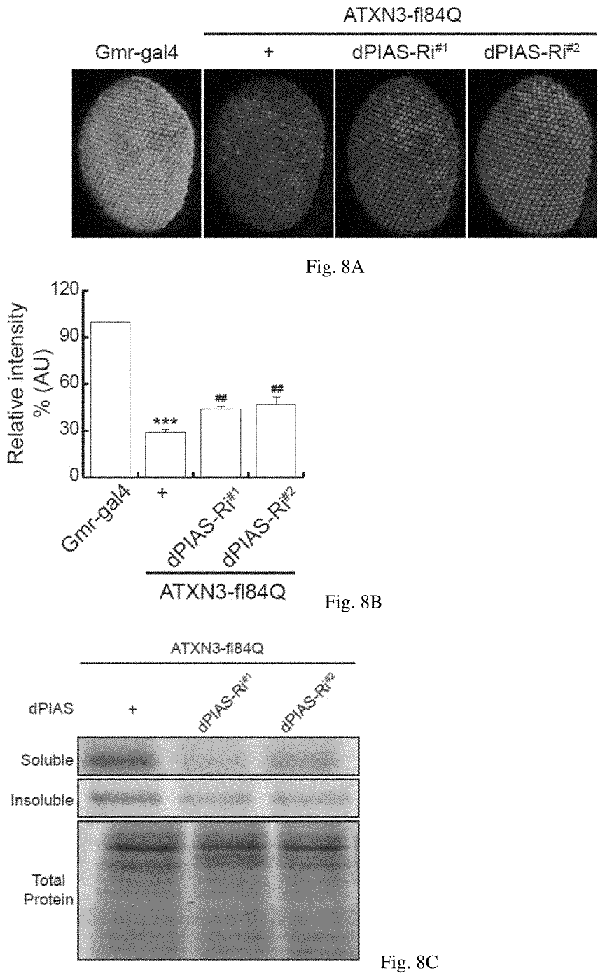

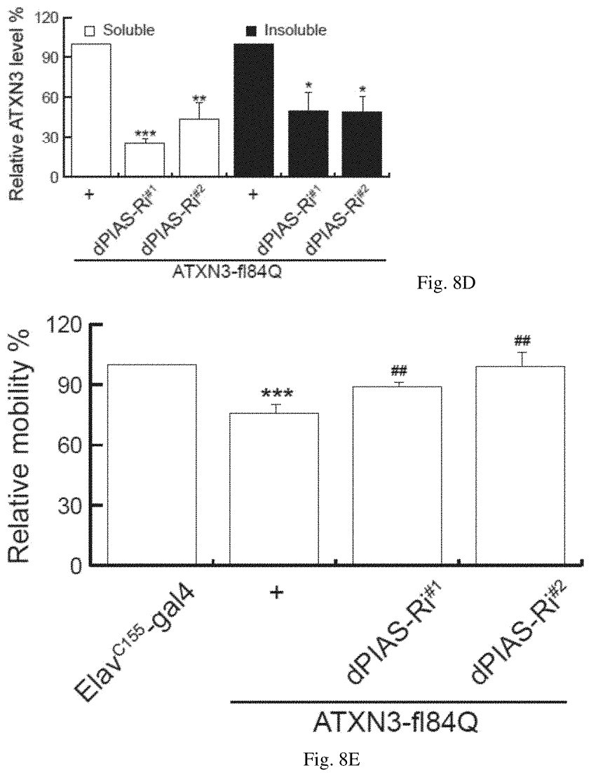

Drosophila overexpressing full-length ATXN3-84Q driven by retinal specific Gmr-gal4 was used as a SCA3 model system, and the membrane-bound mCD8-GFP was used to quantify the degree of degeneration. Overexpression of ATXN3-84Q significantly reduced the expression of mCD8-GFP, while down-regulation of dPIAS by overexpressing dPIAS-RNAi increased the expression of mCD8-GFP (FIG. 8s (A) and (B)), indicating that ATXN3-84Q induced neurotoxicity could be attenuated by reducing the expression of dPIAS. Additionally, knocking down dPIAS reduced the levels of both soluble and insoluble ATXN3-84Q proteins as revealed by quantitative immunoblotting (FIGS. 8 (C) and (D)). Using a negative geotaxis assay, we found that the motor function of SCA3 fly model expressing ATXN3-84Q was improved by silencing the expression of dPIAS (FIGS. 8 (E)).

### Example 6 Identification of Genetic Modifier(s) in LTA Patients with HD or SCA3

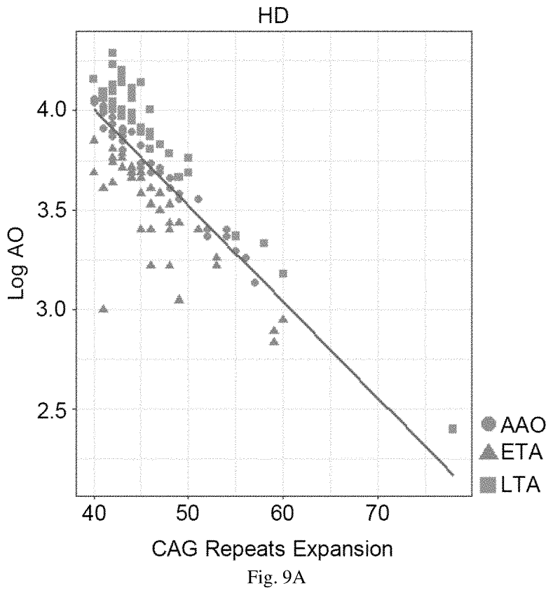

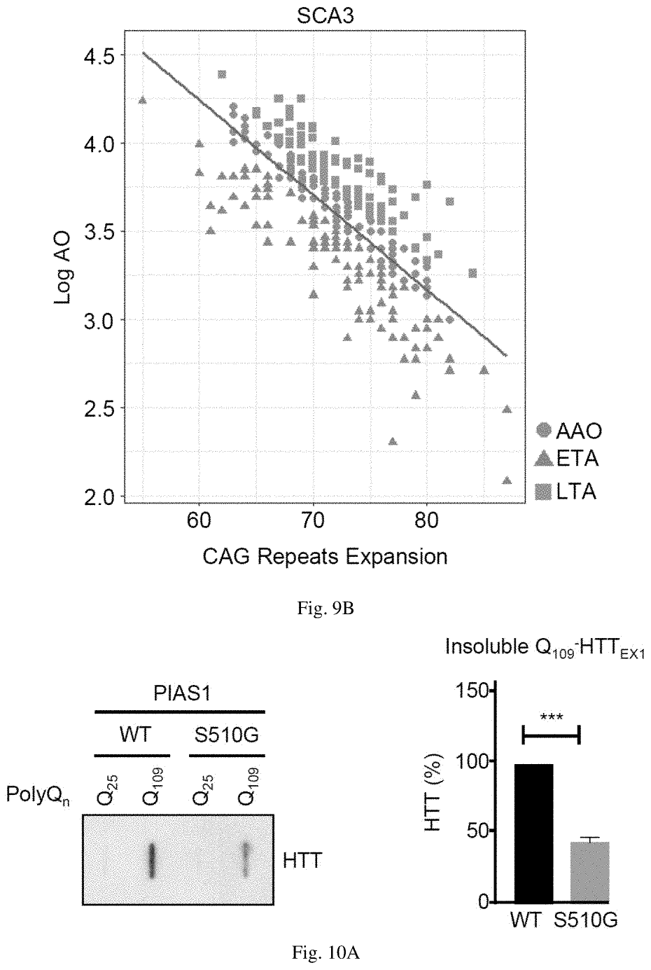

To identify new genetic modifiers for polyQ diseases, we recruited 127 HD and 210 SCA3 patients, and carried out targeted sequencing of 583 genes that are involved in six protein homeostasis pathways and the mTOR signaling pathway. For each disease cohort, a linear regression model was established based on the number of CAG repeats in each disease. The natural logarithm of patients' AO is presented in FIGS. 9 (A) to (B). To explore gene variants across different AO groups, patients were allocated into three groups with similar sample sizes based on residuals from the regression model. The group consisting of patients with an onset time earlier than the average was designated ETA (with only negative residuals), while those with an AO later than the average were designated LTA (with only positive residuals). The group comprising patients with onset at the average AO was designated AAO. Variants identified only in the ETA- or LTA-patients were defined as ETA- or LTA-only variants, respectively. Next, we performed a gene-based chi-squared test to assess whether there was a statistically significant difference between patients with or without ETA-/LTA-only variants among different groups. Because the test was performed in a genewise fashion, a Benjamini-Hochberg correction was used for multiple comparison correction. Genes with corrected p-values smaller than 0.005 were selected as candidate ETA or LTA genes. Among LTA genes in both the HD and/or SCA3 cohorts, PIAS1 was the only gene to have been previously associated with HD (Lee et al., Human Molecular Genetics. 2017; 26(19):3859-67). In total, five candidates of PIAS1 gene variants were found. Two of the PIAS1 gene variants are associated with no change in amino acid residues. We thus chose the three missense PIAS1 variants (N445T, S510G, and T635M) for the following functional validation. Notably, all three PIAS1 variants are located in the C-terminus of PIAS1.

### Example 7 Expression of a PIAS1 Gene Variant (PIAS1^{S510G}) Reduced the Accumulation of mHTT

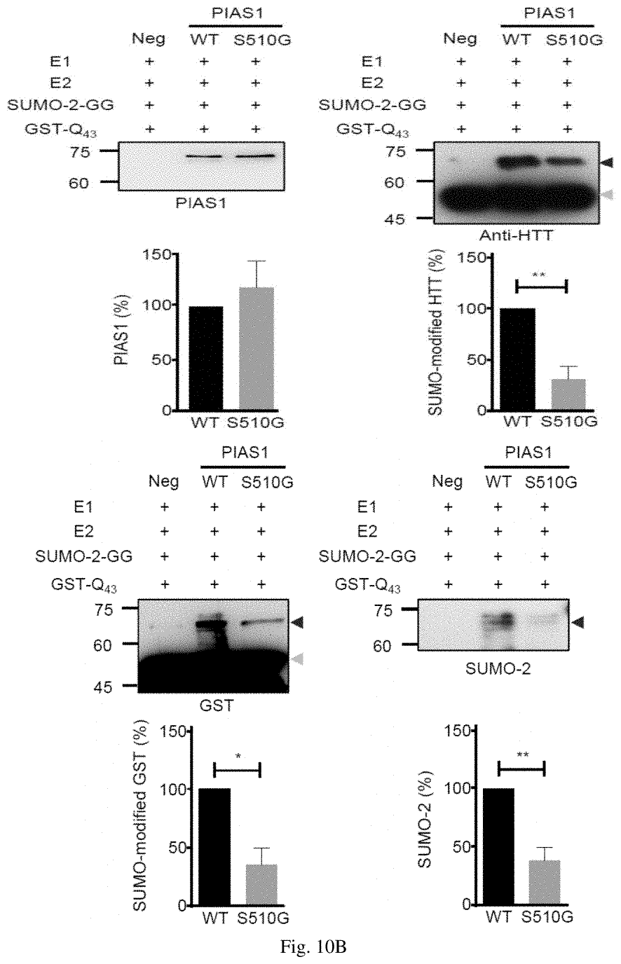

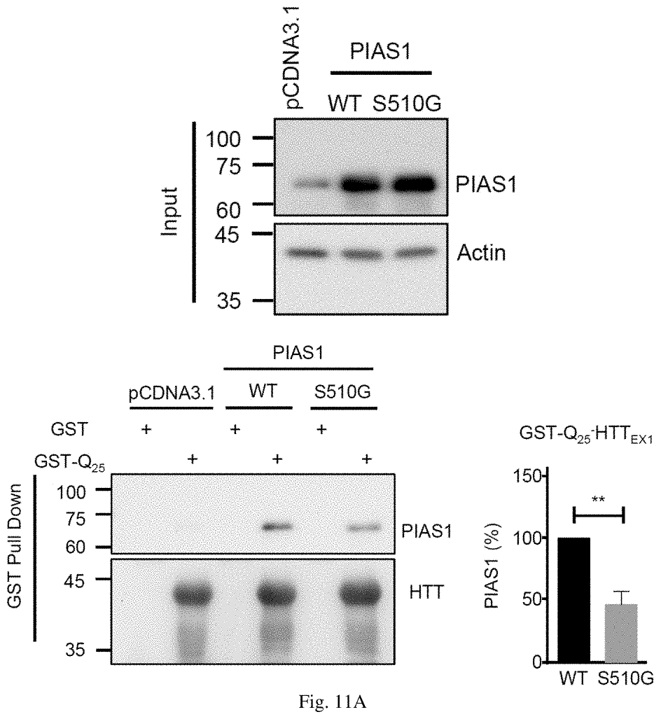

Expression constructs of PIAS1 gene variants were prepared and transiently transfected into HEK293T cells for 48 hrs. Western blot analyses revealed that all three PIAS1 variants (N445T, S510G, and T635M) were successfully expressed in HEK293T cells. Because PIAS1 is known to promote the posttranslational modification of mHTT with SUMO and to mediate the formation of insoluble mHTT, we hypothesized that the ability of these PIAS1 variants to mediate SUMOylation of mHTT may be different than that of wild-type PIAS1 and thus may affect the stability of mHTT in cells. We first assessed the impact of PIAS1 variants on the accumulation of insoluble mHTT in cells using a filter trap assay. Our results showed that PIAS1^{S510G} but not the other two variants tested, significantly reduced the accumulation of insoluble mHTT compared with that of wild-type PIAS1 (FIG. 10 (A)). PIAS1^{S510G} thus was chosen for further analysis.

### Example 8 PIAS1^{S510G} Showed Diminished SUMOylation of mHTT Because of its Defective Interaction with HTT

To assess the effect of PIAS1^{S510G} on the SUMOylation of mHTT, we carried out an in vitro SUMOylation assay of GST-Q₄₃-HTT_{EX1}. Purified components of a SUMOylation reaction (E1, E2, and SUMO-2-GG) were incubated with purified GST-Q₄₃-HTT_{EX1} as the substrate and the corresponding PIAS1 variant (WT or S510G) as the E3. The addition of PIAS1^{WT} enhanced the SUMOylation of GST-Q₄₃-HTT_{EX1} (designated GST-Q₄₃), which was evident by the shift of the GST-Q₄₃-HTT_{EX1} protein to a higher molecular weight in the Western blot analysis (FIG. 10 (B)). Compared with PIAS1^{WT}, PIAS1^{S510G} induced a much lower level of SUMOylation of GST-Q₄₃HTT_{EX1}.

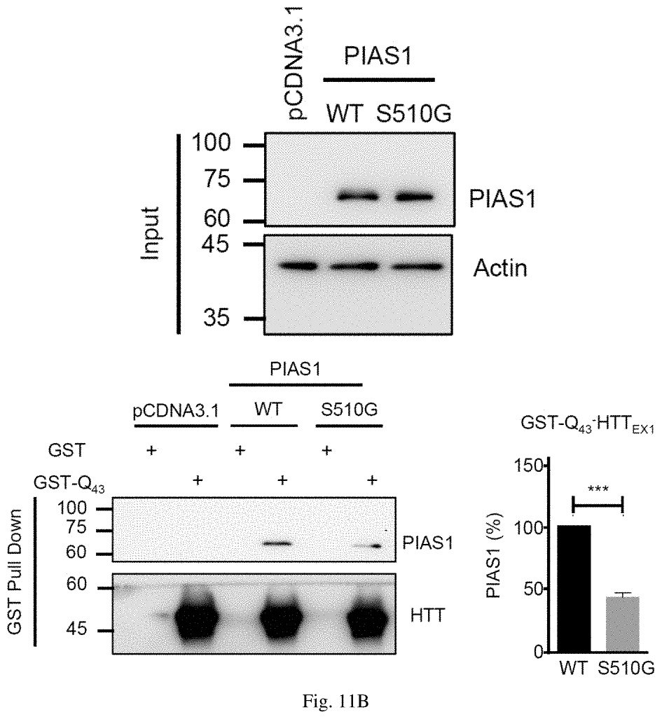

Since binding of PIAS1 to its target substrates is essential to promote SUMOylation, we next examined the binding of PIAS1 to GST-Q₂₅-HTT_{EX1} (designated GST-Q₂₅) or GST-Q₄₃ by GST pull-down assay. Our data revealed that both GST-Q₂₅ and GST-Q₄₃ (but not GST) interacted with PIAS1 (FIGS. 11 (A) to (B)). Most importantly, the ability of PIAS1^{S510G} to interact with either GST-Q₂₅ or GST-Q₄₃ was much lower than that of PIAS1^{W}T. These data collectively suggest that the impaired ability of PIAS1^{S510G} to SUMOylate mHTT likely results from its poor capacity to interact with mHTT.

### Example 9 the S/T-Rich Region of PIAS1 was Critical for its Interaction with HTT

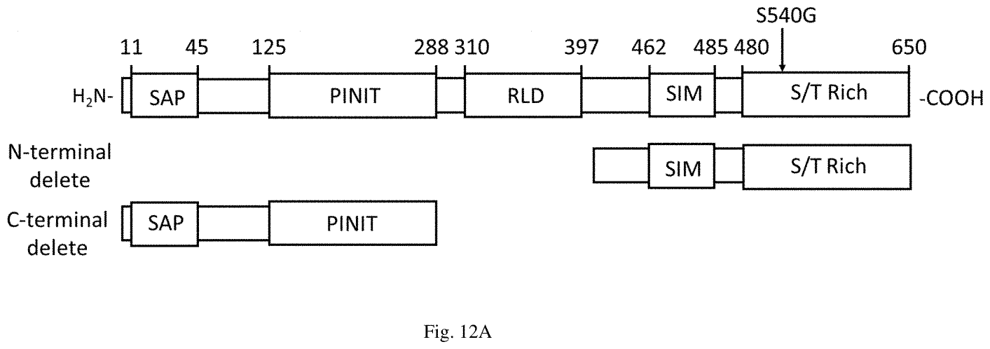

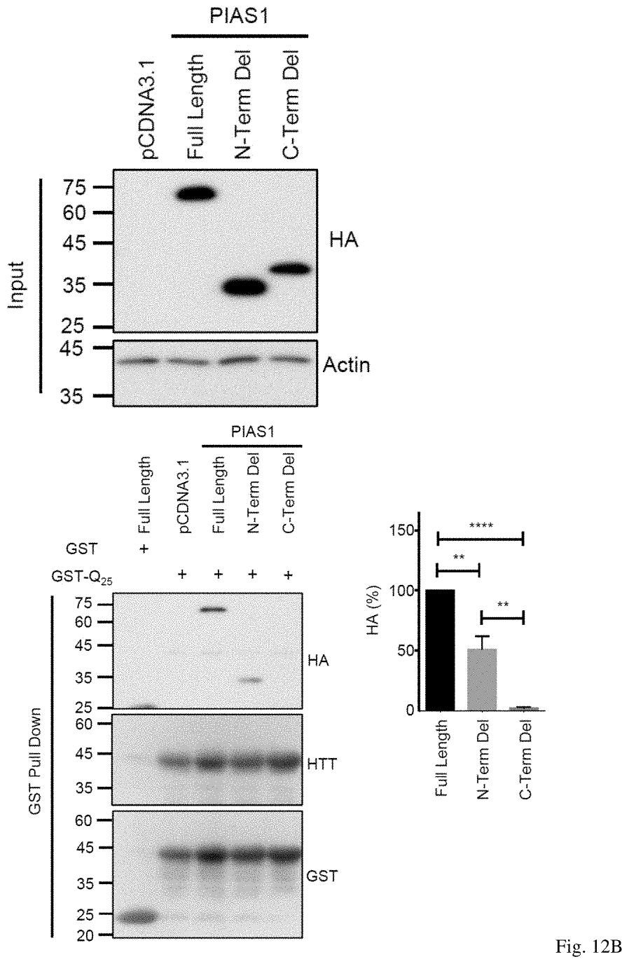

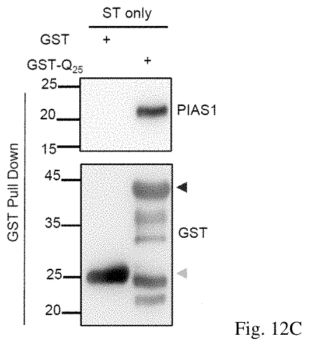

Previous studies have shown that via distinct regions of PIAS1 (Shuai et al., Nature Review Immunology. 2005; 5:593-605), PIAS1 has diverse interacting partners. In particular, PIAS1 serves as an adaptor that interacts with both ubiquitin-conjugating enzyme 9 (Ubc9, an E2 ligase) and its target substrate for SUMOylation (Tozluoglu et al., PLoS Computational Biology. 2010; 6(8), e1000913). It has been previously reported that PIAS1 binds to Ubc9 through its RLD domain (Mascle et al., Journal of Biological Chemistry. 2013; 288(51):36312-27). Considering that Ubc9 may also form direct interactions with target substrates of SUMO conjugation, we created two HA-tagged PIAS1 mutants lacking the RLD domain to determine the domain of PIAS1 that interacts with HTT (FIG. 12 (A)). The GST pull-down assay demonstrated that deletion of the C-terminus (i.e., C-terminal deletion), comprising the RLD and the S/T-rich region, disabled PIAS1 binding to HTT. In contrast, the PIAS1 mutant containing only the SIM and S/T-rich regions (i.e., the N-terminal deletion) was sufficient to bind HTT (FIG. 12 (B)).

To determine whether PIAS1 directly interacts with HTT via the S/T-rich domain, the recombinant PIAS1 mutant that contained only the S/T-rich region (designated S/T only) was tested by pull-down assay for its ability to interact with GST-Q₂₅. Our results showed that GST-Q₂₅, but not GST, pulled down S/T-only PIAS1 (FIG. 12 (C)). Collectively, these results support the notion that the S/T-rich region of PIAS1 is attributable for the protein-protein interaction between PIAS1 and HTT.

### Example 10 Alteration of Ser⁵¹⁰ in the S/T-Rich Region of PIAS1 Affected its Interaction with HTT

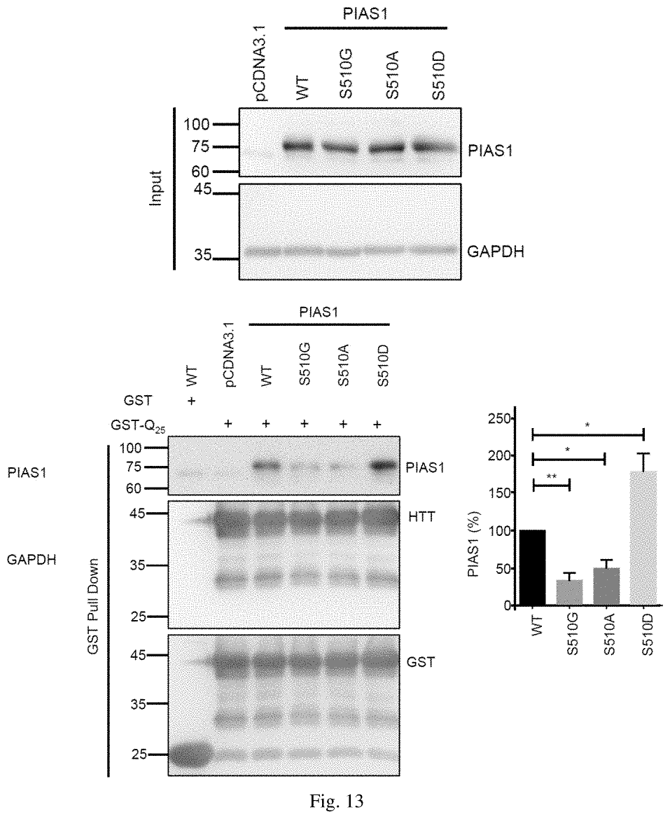

Protein phosphorylation allows dynamic regulation of various biological processes, including protein activity, subcellular localization, stability and protein-protein interactions (Marrero et al., ACS Omega. 2021; 6(8):5091-100). A previous study reported that phosphorylation of Ser⁵¹⁰ in PIAS1 modulates its SUMOylation ability (Cai et al., Circulation Research. 2016; 119(3):422-33). Hence, to assess whether phosphorylation of PIAS1 at Ser⁵¹⁰ plays any role in regulating the SUMOylation of HTT by PIAS1, we substituted Ser⁵¹⁰ with either alanine (phosphorylation-deficient) or aspartic acid (phosphor-mimetic) and assessed the effects of these changes on the ability of PIAS1 to interact with HTT. Similar to PIAS1^{S510G}, the results of the GST pull-down assay indicated that PIAS1^{S510A} had a diminished ability to interact with GST-Q₂₅ (FIG. 13). In contrast, the phosphor-mimetic mutant (PIAS1^{S510D}) had a greater ability to bind to GST-Q₂₅ than PIAS1^{WT}. These results suggest that phosphorylation of PIAS1 at Ser⁵¹⁰ plays an important role in regulating its interaction with HTT.

### Example 11 Knock-In of Pias1^{S510G} Modified the Disease Phenotypes and Lifespan of HD Mice (R6/2)

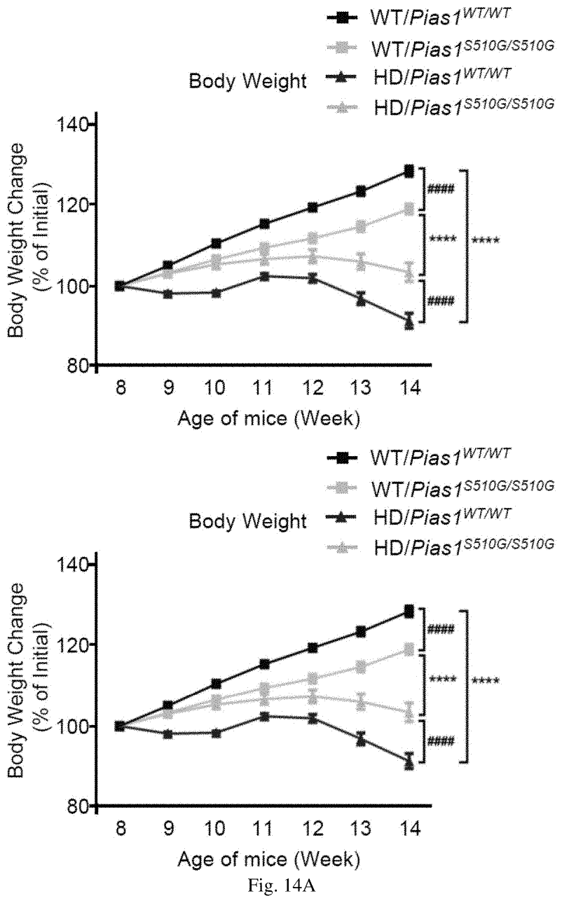

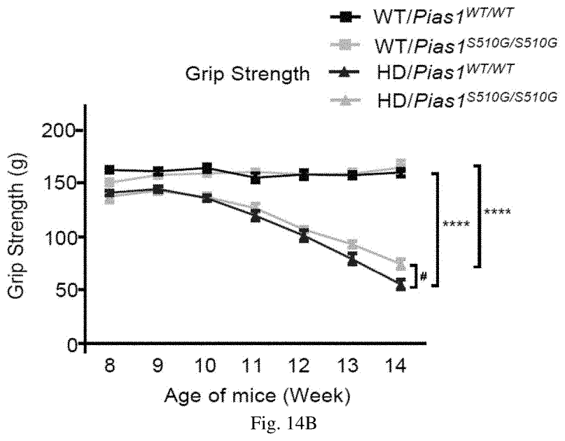

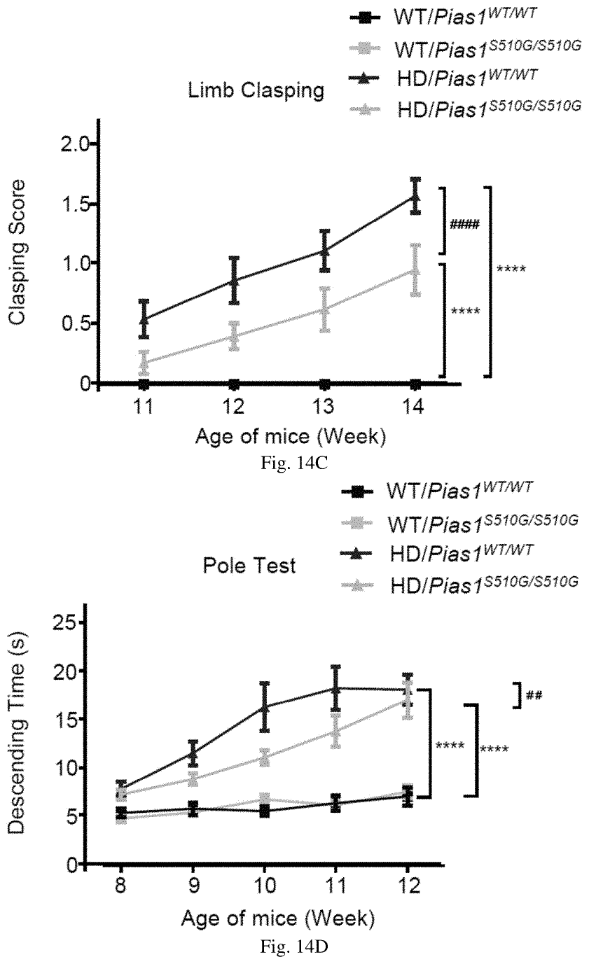

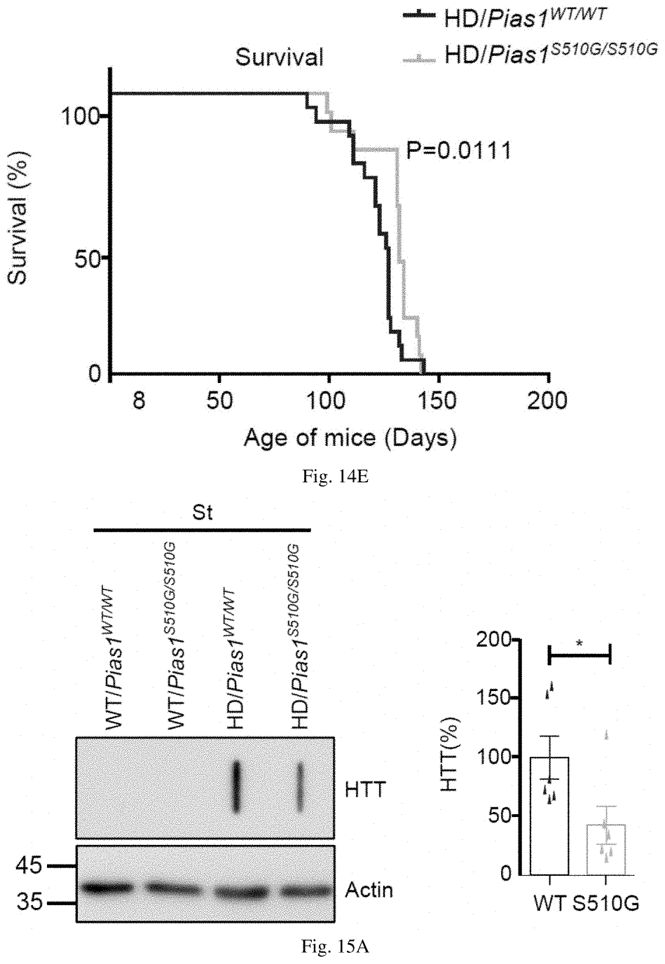

The Ser⁵¹⁰ residue is conserved between humans and mice. Using the CRISPR/Cas9 technique, we therefore generated an HD mouse model (based on R6/2) in which endogenous wild-type Pias1 was replaced with Pias1^{S510G} to study the functional relevance of PIAS1^{S510G} in HD. R6/2 mice were chosen because they recapitulate many symptoms (including progressive weight loss, dystonia, poor motor coordination, and general weakness) of patients with HD. As shown in FIG. 14 (A), the body weight loss in the HD/Pias1^{S510G/S510G} mice was evident in mice 10 weeks old and older, much later than that in the HD/Pias1^{WT/WT} mice. The deterioration of muscle strength assessed by the grip strength assay (FIG. 14 (B)), limb clasping assay (FIG. 14 (C)), and impaired balance assay (i.e., vertical pole test; FIG. 14 (D)), were less severe in the HD/Pias1^{S510G/S510G} mice than in the HD/Pias1^{WT/WT} mice. Nonetheless, no changes in locomotor activity or rotarod, beam walking test or Y-maze performances were observed between the HD/Pias1^{S510G/S510G} and HD/Pias1^{WT/WT} mice. Importantly, the survival of the HD/Pias1^{S510G/S510G} mice was longer than that of the HD/Pias1^{WT/WT} mice (FIG. 14 (E)).

### Example 12 Expression of Pias1^{S510G} Modulated the SUMO Modification and Accumulation of mHTT in HD Mice

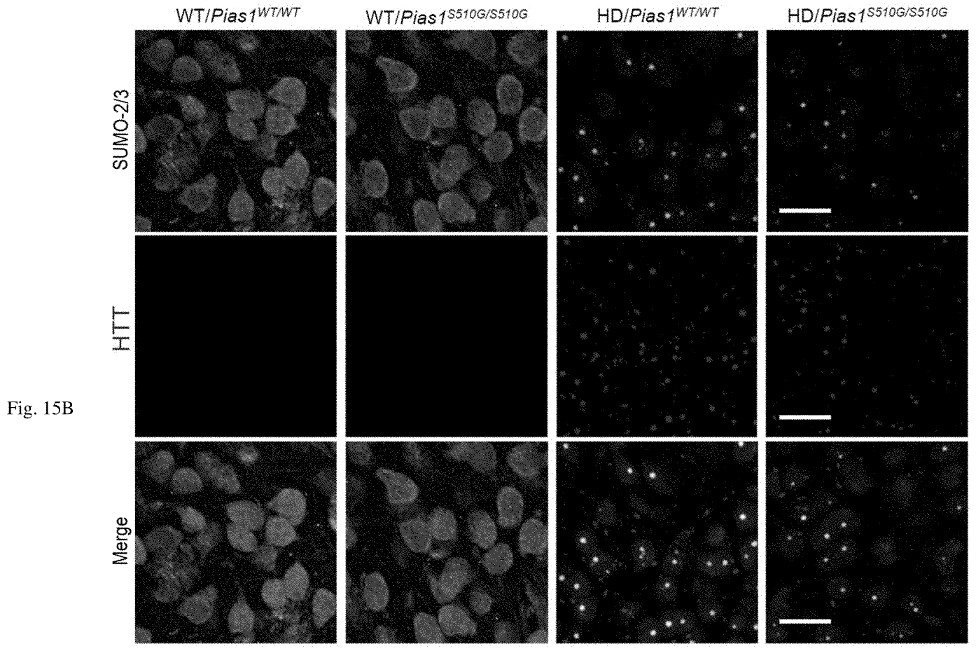

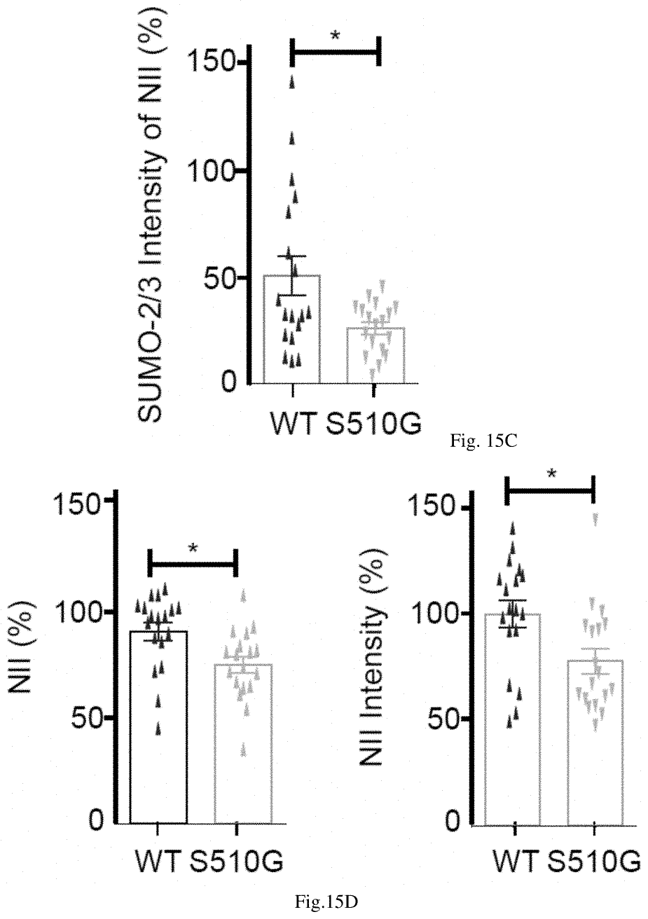

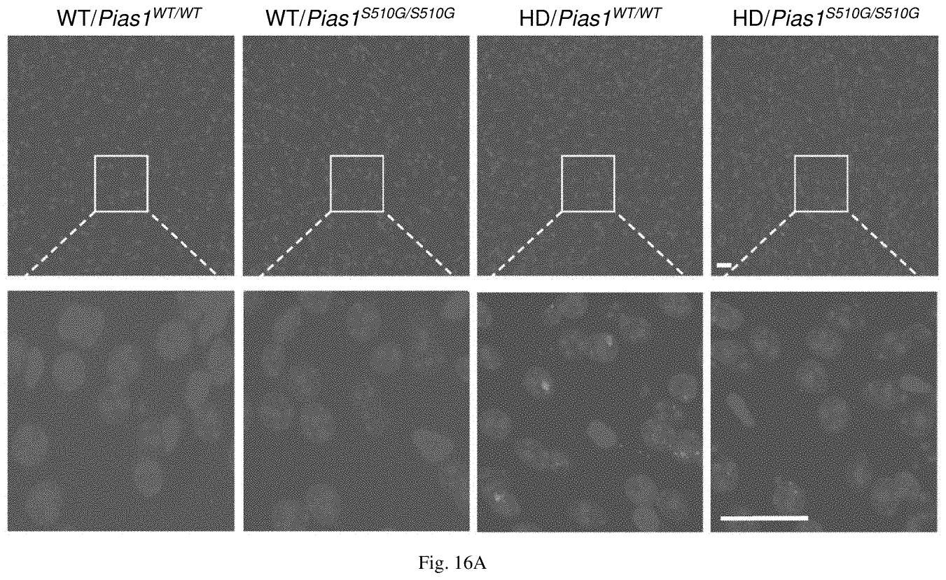

A neuropathological hallmark of HD is the accumulation of insoluble mHTT (DiFiglia et al., Neuron. 1995; 14:1075-81). We next analyzed the accumulation of mHTT aggregates in vivo in mice 13 weeks old (late HD stage), where widespread neuronal intranuclear inclusions of mHTT can be readily observed. The results of the filter trap assay demonstrated that the expression of Pias1^{S510G} led to reduced accumulation of insoluble mHTT in the striatum of the HD mice (FIG. 15 (A)). Immunofluorescence staining of SUMO-2/3 and mHTT revealed diffuse subcellular localization of SUMO-2/3 in both the nucleus and cytoplasm of the WT brains, while SUMO-2/3 was colocalized with neuronal intranuclear mHTT inclusions (FIG. 15 (B)). Quantitative analysis showed that the level of intranuclear inclusions with SUMO conjugation was significantly lower in the HD/Pias1^{S510G/S510G} mice than in the HD/Pias1^{WT/WT} mice (FIG. 15 (C)). Consistent with the results of the filter trap assay, both the amount and average intensity of neuronal intranuclear mHTT inclusions were significantly lower in the HD/Pias1^{S510G/S510G} mice than in the HD/Pias1^{WT/WT} mice. (FIG. 15 (D)).

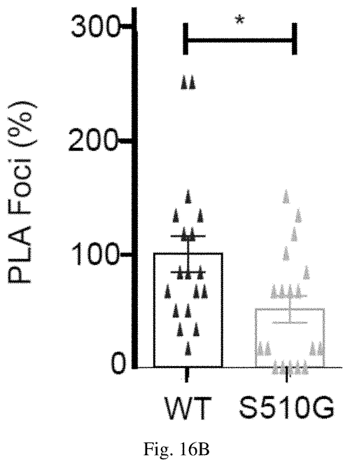

We further carried out an in situ proximity ligation assay (PLA, (DiFiglia et al., Neuron. 1995; 14:1075-81, 39)) to evaluate endogenous SUMO-modified mHTT levels using anti-SUMO-2/3 and anti-HTT antibodies. PLA signals were observed only in the brain sections of the HD mice, not in those of the WT mice, suggesting that PLA signals were specific to SUMO-modified mHTT (FIG. 16 (A)). Substantially fewer PLA signals were found in the striatum of the HD/Pias1^{S510G/S510G} mice than in the HD/Pias1^{WT/WT} mice (FIG. 16 (B)), in agreement with our in vitro analysis showing that Pias1^{S510G} reduced the extent of SUMO-modification and mHTT accumulation.

While the present disclosure has been described in conjunction with the specific embodiments set forth above, many alternatives thereto and modifications and variations thereof will be apparent to those of ordinary skill in the art. All such alternatives, modifications and variations are regarded as falling within the scope of the present disclosure.

What is claimed is:

1. A method of identifying a genetic modifier of a nucleotide repeat disorder, comprising: (a) providing the length of one or more nucleotide repeats in samples obtained from subjects to obtain nucleotide repeat load in genomes of the subjects; (b) clustering the subjects based on overall nucleotide repeat loads in the subject; (c) selecting from the subjects the late-onset subjects with higher nucleotide repeat load or the early-onset subjects with lower nucleotide repeat load; and (d) identifying one or more genetic modifiers delaying or promoting onset of a nucleotide repeat disorder.

2. The method of claim 1, wherein the nucleotide repeat is a CGG-repeat, a CTG-repeat, a GAA-repeat or a CAG-repeat.

3. The method of claim 1, wherein the nucleotide repeat disorder is Huntington's disease (HD), spinocerebellar ataxias, spinal and bulbar muscular dystrophy (SBMA), or dentatorubral-pallidoluysian atrophy (DRPLA).

4. The method of claim 1, wherein the nucleotide repeat load is a CAG load, a CGG load, a CTG load or a GAA load.

5. A computer system for performing the method of identifying a genetic modifier of a nucleotide repeat disorder as claimed in claim 1, comprising:
a database that is configured to store data of the length of one or more nucleotide repeats in samples obtained from subjects to obtain nucleotide repeat load in genomes of the subjects; and
one or more computer processors operatively coupled to the database, wherein the one or more computer processors are individually or collectively programmed to cluster the subjects based on overall nucleotide repeat loads in the subject; select from the subjects the late-onset subjects with higher nucleotide repeat load or the early-onset subjects with lower nucleotide repeat load; and identify one or more genetic modifiers delaying or promoting onset of a nucleotide repeat disorder.

6. A PIAS1 variant or mutant or a recombinant nucleic acid molecule encoding a PIAS1 variant or mutant, wherein the PIAS1 variant or mutant comprises one or more sequence changes located in the C-terminal region of PIAS1.

7. The PIAS1 variant or mutant or a recombinant nucleic acid molecule encoding the PIAS1 variant or mutant of claim 6, wherein the sequence change is S510G, A445T or T635M or one or more combinations thereof.

8. A method for treating or preventing a polyglutamine (polyQ) expansion disease in a subject in need of such treatment or prevention, comprising administering an effective amount of the PIAS1 variant or mutant or a recombinant nucleic acid molecule encoding the PIAS1 variant or mutant of claim 6 to the subject.

9. The method of claim 8, wherein the PIAS1 variant or mutant is selected from S510G, A445T and T635M and one or more combinations thereof.

10. The method of claim 8, wherein the method is for reducing accumulation of mutant polyQ proteins, for preventing mutant polyQ proteins aggregation and toxicity, for lowering SUMOylation of mutant polyQ proteins, for de-stabilizing mutant polyQ proteins, for decrease of SUMO3-conjugation on mutant polyQ proteins, for reduction of foci formation and cell death, for improving motor function, and/or for treating or preventing early symptoms onset of the polyglutamine expansion disease in the subject.

11. The method of claim 10, wherein the polyQ proteins are huntingtin (HTT), ataxin-1 (ATXN), ataxin-2 (ATXN2), ataxin-3 (ATXN3), calcium voltage-gated channel subunit alphal A (CACNA1A), ataxin-7 (ATXN7), TATA box-binding protein (TBP), atrophin-1 (ATN1), or androgen receptor (AR).

12. The method of claim 8, wherein the polyglutamine expansion disease is Huntington's disease, spinocerebellar ataxias, spinal and bulbar muscular dystrophy, or dentatorubral-pallidoluysian atrophy.

13. A method for treating or preventing early symptoms onset of the polyglutamine expansion disease in a subject in need of such treatment or prevention, comprising administering an effective amount of an agent for diminishing the effect of wild type PIAS1 in the subject.

14. The method of claim 13, wherein the agent is a PIAS1 variant or mutant or a recombinant nucleic acid molecule encoding the PIAS1 variant or mutant to the subject, wherein the PIAS1 variant or mutant comprises one or more sequence changes located in the C-terminal region of wild type PIAS1.

15. The method of claim 14, wherein the PIAS1 variant or mutant is selected from S510G, A445T and T635M and one or more combinations thereof.

16. The method of claim 13, wherein the agent is an RNA interference agent (RNAi).

17. The method of claim 16, wherein the RNAi is a small inhibitory RNA (siRNA), a microRNA (miRNA), or a small hairpin RNA (shRNA).

18. The method of claim 13, wherein the method is for reducing accumulation of mutant polyQ proteins, for preventing mutant polyQ proteins aggregation and toxicity, for lowering SUMOylation of mutant polyQ proteins, for de-stabilizing mutant polyQ proteins, for decrease of SUMO3-conjugation on mutant polyQ proteins, for reduction of foci formation and cell death, for improving motor function in the subject.

19. The method of claim 18, wherein the polyQ proteins are HTT, ATXN1, ATXN2, ATXN3, CACNA1A, TBP, ATN1, or AR.

20. The method of claim 13, wherein the polyglutamine expansion disease is Huntington's disease, spinocerebellar ataxias, spinal and bulbar muscular dystrophy, or dentatorubral-pallidoluysian atrophy.
